(19) United States
(12) Patent Application Publication
(10) Pub. No.: US 2024/0043523 A1
(43) Pub. Date: Feb. 8, 2024

(54) METHODS OF SELECTIVELY REDUCING ANTIBODIES

(71) Applicant: FRESENIUS KABI DEUTSCHLAND GMBH, Bad Homburg (DE)

(72) Inventors: Mathilde CHAPUIS, Bad Homburg (DE); Cecilia COURBEY, Bad Homburg (DE); Vincent DECHAVANNE, Bad Homburg (DE); Simon FRADIN, Bad Homburg (DE); Coralie BORROSSI, Bad Homburg (DE); Periklis SELEVOS, Bad Homburg (DE)

(21) Appl. No.: 18/255,481

(22) PCT Filed: Dec. 2, 2021
(86) PCT No.: PCT/EP2021/084073

§ 371 Date: Jun. 1, 2023

(30) Foreign Application Priority Data
Dec. 2, 2020 (GB) 2019023.7
Jun. 16, 2021 (GB) 2108596.4

(51) Int. Cl. C07K 16/24; B01D 15/36; B01D 15/20; C12N 5/071

(52) U.S. Cl. CPC C07K 16/244; B01D 15/362; B01D 15/203; C12N 5/0682; C07K 2317/14; C12N 2500/34; C07K 2317/565; C12N 2501/999

(57) ABSTRACT

The invention relates to upstream and/or downstream processes of selectively reducing one or more unpaired cysteines of a monoclonal antibody, whilst keeping conserved inter- and intra-molecular disulfide bonds elsewhere in the antibody intact. The invention further relates to purified antibodies obtained by the methods as described herein.

### FIELD OF THE INVENTION

The present invention relates to upstream and/or downstream processes of selectively reducing one or more unpaired cysteines of a monoclonal antibody, whilst keeping conserved inter- and intra-molecular disulfide bonds elsewhere in the antibody intact. The invention further relates to purified antibodies obtained by the methods as described herein.

### BACKGROUND TO THE INVENTION

The commercial production of monoclonal antibodies has revolutionized the treatment of many diseases. Typical IgG1 antibodies comprise two light chains (L) and two heavy chains (H), with molecular weights of roughly 25 KDa and 50 KDa, respectively. The light chain and heavy chain are connected by a single inter-chain disulfide bond (L-S—S—H). The two LH units are further connected by two inter-chain disulfide bridges between the heavy chains. Consequently, the general formula for classical IgG1 antibodies is L-SS—H(—SS₂—)—H—SS-L.

In addition, monoclonal antibodies typically possess highly conserved intra-chain disulfide bonds. Both inter- and intra-disulfide bonds are vital for the correct structure and function of an antibody. Formation of disulfide bonds typically occurs from highly conserved free cysteine residues which spontaneously form an —S—S— bond. Formation of disulfide bonds is dependent upon the spatial orientation of the free cysteines, the environmental redox potential, the environmental pH, and the presence of thiol oxidizing enzymes.

Some atypical monoclonal antibodies contain unpaired cysteines that do not partake in cognate disulfide bond formation but are instead required for antigen recognition and binding. Maintaining the free status of unpaired cysteines may be required to maintain the stability and activity of the antibody. For example, blocking (e.g. oxidation or mispairing) of unpaired cysteines may occur during large-scale monoclonal antibody production resulting in misfolded, de-natured and/or inactive protein.

By way of example, anti-IL-17 antibodies such as secukinumab have an unpaired cysteine at position 97 of the amino acid light chain (LC Cys97). This unpaired cysteine is located within the light chain complementarity determining region 3 (LCDR3) of secukinumab. To retain biological activity, the unpaired Cys97 should not be blocked by oxidation from endogenous compounds or oxidative disulfide pairing with other cysteines. During recombinant production by mammalian cell lines, such undesirable oxidative modifications may frequently occur. Consequently, Cys97 needs to be unblocked (e.g., un-cysteinylated) to allow activity of the antibody, whilst avoiding reduction of conserved intra-chain and inter-chain disulfide bonds elsewhere leading to fragmentation of the antibody.

Existing methods for selective reduction of secukinumab may involve contacting the antibody with reducing agents such as cysteine to a bioreactor whilst actively maintaining dissolved oxygen levels under controlled parameters (see, for example, US2017/0369567A1). For selective reduction of Cys97 to occur in such techniques, the level of oxygen in the bioreactor needs to be kept low during the incubation step. However, this method requires strict regulation of oxygen levels. Furthermore, maintenance of oxygen transfer may vary greatly depending upon the apparatus utilized, requiring significant optimization and use of non-standard equipment. Additionally, additional step(s) of downstream quenching of excess reducing agent are required. Such techniques also require additional steps of adding reducing agents such as cysteine to the antibody mixtures, increasing the cost of the process.

It is an aim of some embodiments of the present invention to mitigate some of the problems identified in the prior art.

### SUMMARY OF CERTAIN EMBODIMENTS OF THE INVENTION

The invention relates to methods for selectively reducing one or more unpaired cysteines of a monoclonal antibody during upstream and/or downstream processing steps.

In certain embodiments, the invention provides a method of selectively reducing one or more unpaired cysteines of a recombinant monoclonal antibody (e.g., secukinumab or variant thereof) during production of the antibody, wherein the method comprises:
(a) providing a cell (e.g., a CHO cell) capable of recombinant expression of the antibody;
(b) culturing the cell in a cell culture medium, wherein the cells are cultured at a first temperature (e.g., 37° C.) and then shifted to a second temperature, wherein the second temperature is lower than the first temperature (e.g., 33° C.),
wherein the cells are maintained in culture until at least 90% or more of the unpaired cysteines are de-cysteinylated (e.g., for between about 14 to about 17 days); and
(c) harvesting the antibodies from the cell culture to obtain a preparation of the antibody.

In certain embodiments, the control of pH is also optimized during cell culture to facilitate selective reduction of the unpaired cysteines of the antibodies during production. Typically, the pH of the cell culture is maintained, e.g., at a pH of between about 6.7 to 7.1, optionally until at least 90% or more of the unpaired cysteines are de-cysteinylated (e.g., for between about 14 to about 17 days).

Advantageously, such methods provide an efficient method of producing selectively reduced antibodies with high levels of de-cysteinylation during upstream processing without the need for addition of any reducing agents and/or any other steps to increase de-cysteinylation during downstream processing.

The methods of the invention are simple, cost-effective and provide highly stable monoclonal antibodies that fully retain biological activity and structure as compared to reference approved antibodies.

As further described herein, the control of temperature is optimized during cell culture to selectively reduce the unpaired cysteines of the antibodies during production. Typically, the temperature is actively shifted from a first temperature to a second temperature during cell culture. For example, the second temperature may be between about 3° C. to about 5° C. lower than the first temperature. Preferably, the second temperature is about 4° C. lower than the first temperature.

In preferred embodiments, the first temperature is about 37° C. and the second temperature is about 33° C. Typically, the cells are cultured at the first temperature for between about 8 days to about 13 days, preferably about 10 days, before culturing the cells at the second temperature (e.g., up to about 17 days).

In certain embodiments, the total duration of cell culture is optimized to selectively reduce the unpaired cysteines of the antibodies during production. Typically, the cells are maintained in culture until at least 90% or more of the unpaired cysteines are de-cysteinylated. Techniques for determining % cysteinylation of antibodies are well-described in the art and include, for example, hydrophobic interaction chromatography (HIC) or the like.

In preferred embodiments, the cells are cultured for a total period of between about 14 to about 17 days, e.g., at least about 14, 15, 16 or 17 days or more. Typically, such time periods allow at least 90% or more of the unpaired cysteines of the antibodies to be de-cysteinylated.

Advantageously, the optimized upstream processes described herein are capable of producing high % de-cysteinylated antibodies (e.g., about 91%, 92%, 93%, 94%, 95%, 96%, 97%, 98% or more). In such embodiments, addition of any reducing agents and/or further de-cysteinylation during any downstream processing steps may not be required.

In alternative embodiments, non-optimized upstream processes may be used to produce the recombinant monoclonal antibodies having one or more unpaired cysteines. Non-optimized processes may lead to low % de-cysteinylated antibodies (e.g., about 90%, 89%, 88%, 87%, 86%, 85%, 84%, 83%, 82%, 81%, 80% or less). In such scenarios, further de-cysteinylation during any downstream processing steps may be required. As such, the invention also provides methods of on-column reduction to increase de-cysteinylation of the antibodies during downstream processing.

In such alternative embodiments, the invention may comprise contacting harvested antibodies with a mixture comprising one or more reducing agents whilst the antibodies are adsorbed to a chromatography material (e.g., Protein A). Advantageously, such methods avoid the need to regulate dissolved oxygen levels during downstream processing steps. In addition, the selective reduction step can be performed at room temperature avoiding the need to heat the mixture. The “on-column” reduction of antibodies also avoids the need for any extra step of removing the reducing agent. Instead, the reducing agent can simply be eluted following the usual column wash step.

These alternative methods of the invention are also simple, cost-effective and provide highly stable monoclonal antibodies that fully retain biological activity and structure as compared to reference approved antibodies.

Accordingly, the invention also provides a method of selectively reducing one or more unpaired cysteines of a monoclonal antibody (e.g., secukinumab, variant or biosimilar thereof), wherein the method comprises:
(a) contacting a sample of antibodies (e.g., clarified cell culture fluid) with one or more chromatography materials (e.g., protein A) under any suitable conditions that allow binding of the antibodies to the chromatography material;
(b) contacting the bound antibodies with a mixture comprising one or more reducing agents (e.g., cysteine) for any suitable amount of time; and
(c) eluting the antibodies from the chromatography material.

In certain embodiments, the methods of on-column reduction are performed if the harvested antibodies have a low % de-cysteinylation (e.g., about 90%, 89%, 88%, 87%, 86%, 85%, 84%, 83%, 82%, 81%, 80% or less).

As further described herein, the method of the invention may be optimized depending on, for example, the initial % of un-cysteinylated antibodies in the sample being loaded onto the chromatography material. For instance, the molar ratio of the reducing agent:antibody being used and/or the total duration of contact of the reducing agent with the antibody whilst bound to the chromatography material may be adjusted to optimize selective reduction of the unpaired cysteines whilst minimizing fragmentation of the antibody.

In one embodiment, between about 80% to about 90% of the unpaired cysteines in step (a) are un-cysteinylated. In such embodiments, the molar ratio of the reducing agent (e.g. cysteine):antibody (e.g., secukinumab, variant or biosimilar thereof) is preferably between about 20:1 to about 30:1. In addition, the mixture comprising the one or more reducing agents (e.g. cysteine) is preferably contacted with the antibodies for between about 5 to 6 hours.

In one embodiment, antibodies that are eluted from the chromatography material (e.g., protein A) are held under temperature, time and/or buffer conditions that decrease the % low molecular weight fragments (LMW) in the sample. As used herein a “decreased” % of LMW in the sample may refer to a significant decrease in fragmentation of the antibodies as compared to a sample not subjected to any such additional holding step as described herein.

In preferred embodiments, downstream processing steps are optimized to remove any acidic variants and/or glutathionylation from the sample of antibodies (e.g., obtained following the optimized upstream processes of cell culture and/or optional downstream processes of “on-column” reduction as described herein). Typically, downstream processing steps of anion exchange chromatography (AEX), cation exchange chromatography (CEX) and/or multimodal chromatography (MMC) or the like are optimized to remove any acidic variants and/or glutathionylation in the sample of antibodies.

In certain embodiments, the downstream processing steps to remove any acidic variants and/or glutathionylation are performed regardless of whether de-cysteinylation of the antibodies has been achieved by (a) the optimized upstream processes of cell culture as described herein and/or (b) the downstream processes of “on-column” reduction as described herein.

Typically, the downstream processing steps to remove any acidic variants and/or glutathionylation are optimized depending on, for example, the amount of antibody that is loaded onto the chromatography material (e.g., CEX, AEX and/or MMC or the like). Exemplary load densities are in the range from about 1 to about 100 g/L resin, typically about 10 g/L resin or more. Antibodies in the sample are bound to the chromatography material (e.g., CEX or AEX) as a result of this loading step.

In certain embodiments, the loading density of antibodies onto the chromatography material (e.g., CEX, AEX and/or MMC) is about 10 g/L resin or less. In such embodiments, the method of the invention may comprise:
(a) passing the antibodies through one or more chromatography material(s) (e.g., CEX, AEX and/or MMC), thereby binding the antibodies to the chromatography material(s);
(b) contacting the bound antibodies with a wash buffer, wherein the wash buffer is at a pH above the isoelectric point (pI) of the antibodies in the sample (e.g., at a pH between about 8.8 to 9.0); and
(c) eluting the antibodies from the chromatography material.

In alternative embodiments, the loading density of antibodies onto the chromatography material (e.g., CEX, AEX and/or MMC) is more than about 10 g/L resin (e.g., about 20 g/L, 30 g/L, 40 g/L, 50 g/L, 60 g/L, 70 g/L, 80 g/L, 90 g/L or more). In such embodiments, the method of the invention may comprise:
(a) passing the antibodies through one or more chromatography material(s) (e.g., CEX, AEX and/or MMC), thereby binding the antibodies to the chromatography material(s);
(b) contacting the bound antibodies with a wash buffer, wherein the wash buffer is the same or below the pI of the antibodies in the sample (e.g., at a pH between about 7.0 to 8.4, preferably about pH 8.0); and
(c) eluting the antibodies from the chromatography material.

Advantageously, such pH washes act to remove any acidic variants and/or glutathionylated species in the sample of antibodies. Typically, the antibodies eluted from the chromatography material have decreased % of acidic variants and/or glutathionylation as compared to the antibodies previously loaded onto the chromatography material. Preferably, the chromatography material is CEX.

The invention also provides a purified preparation of antibodies (e.g., secukinumab, biosimilar or variant thereof) obtainable by any method as described herein. In preferred embodiments, the antibodies of the invention are at least about 95% or more un-cysteinylated and about 98% or more intact.

### DETAILED DESCRIPTION OF CERTAIN EMBODIMENTS

Embodiments of the invention will now be described by way of example only, and with reference to the accompanying FIGURES in which:

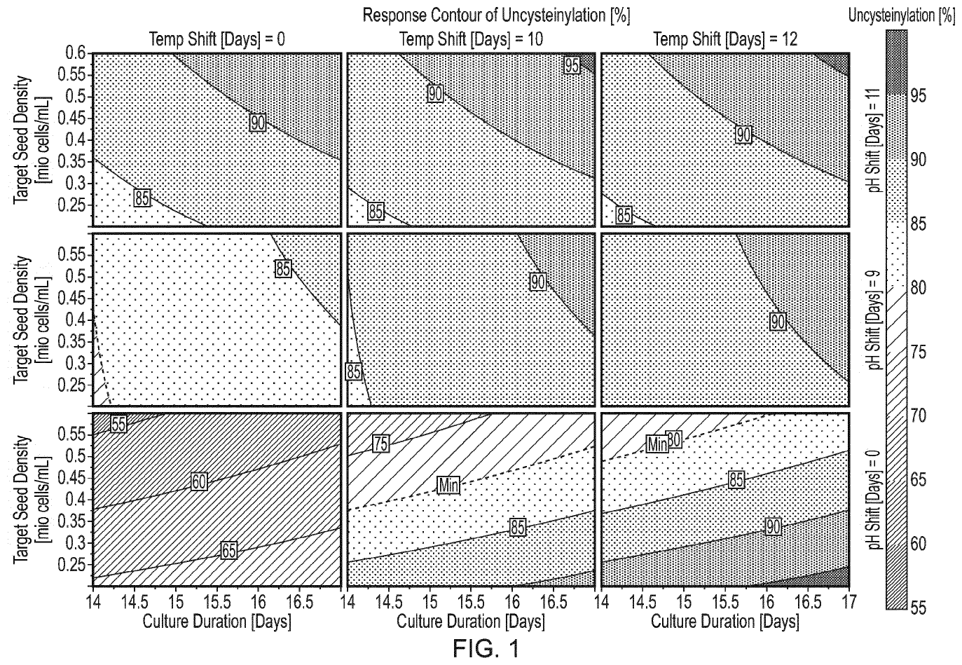

FIG. 1 shows contour plots illustrating the effect of target seed density, temperature shift, pH shift, and culture duration on % un-cysteinylated antibodies during CHO cell culture.

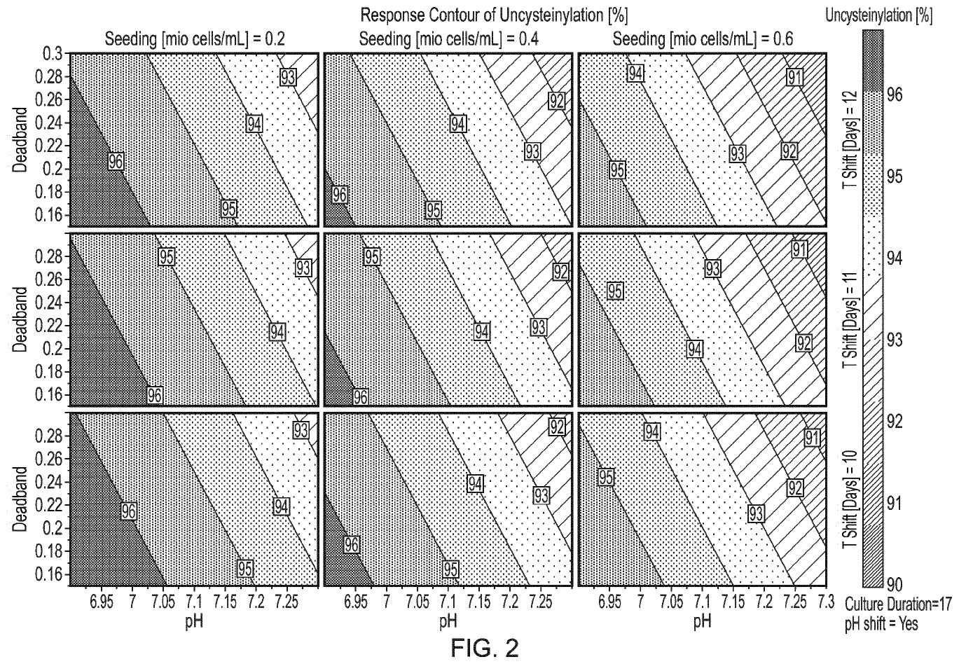

FIG. 2 shows contour plots illustrating the effect of pH set point, pH dead band range, target seed density, and temperature shift on % un-cysteinylated antibodies during CHO cell culture.

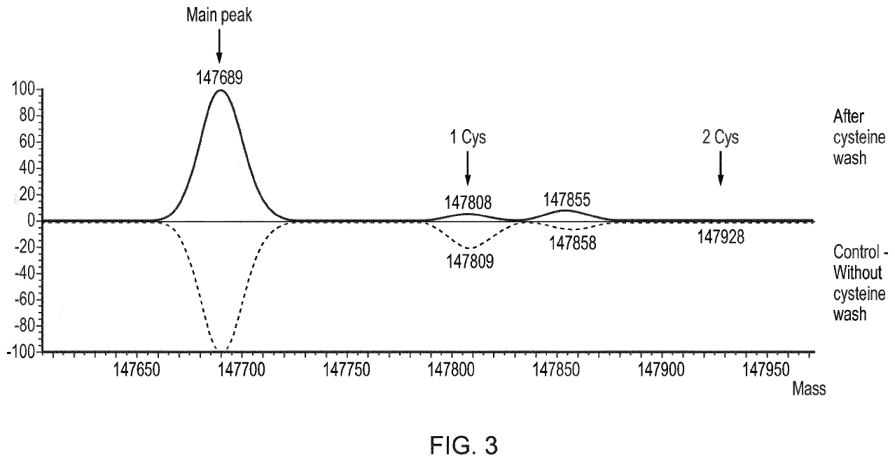

FIG. 3 shows the comparison by LC-MS of two samples after a protein A chromatography step with or without the cysteine wash treatment. The main peak corresponds to the intact antibody free of cysteine, corresponding to a theoretical molecular mass of secukinumab lacking a C-terminal lysine from each heavy chain (i.e. 147688.68 Da). The peak labelled 1 Cys corresponds to the antibody containing one single cysteinylated C97 and one free cysteine. The peak 2 Cys corresponds to the antibody containing two cysteinylated C97.

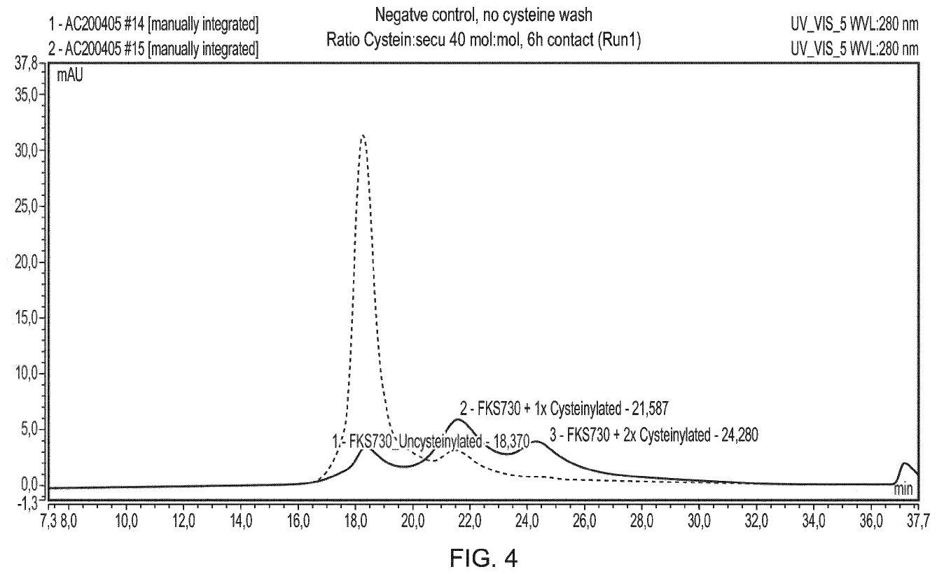

FIG. 4 shows an example of a Hydrophobic Interaction Chromatography-High Pressure Liquid Chromatography (HIC-HPLC) result comparing the profile without cysteine washing (in black, lower peak) and after the cysteine washing (in blue, higher peak). The profile in blue corresponds to run 1 of DOE1 (ratio Cys:mAb of 40:1 and time of static contact 6 hours). The results show that peak 2 (1× cysteinylated) and peak 3 (2× cysteinylated) are decreased in profit of the main peak (de-cysteinylated antibody).

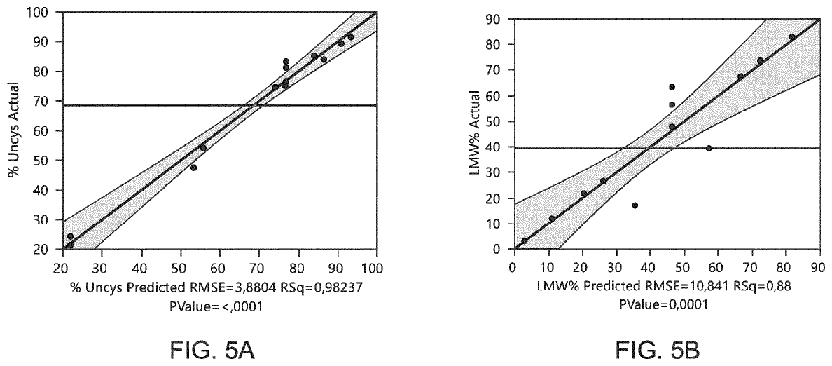

FIG. 5 shows actual vs predicted plots for evaluated responses derived from DOE1 results. FIG. 5A shows a plot for % un-cysteinylated species; FIG. 5B shows a plot for LMW % species. FIG. 5 shows that the points are close to the fitted line with narrow confidence bands, demonstrating a good correlation between the model and the data generated. The mathematical model showed good fit, for both responses, with a R² of 0.98 for the un-cysteinylated species percentage (as measured by HIC-HPLC) and a R² of 0.88 for the LMW species percentage (measured by Capillary Gel Electrophoresis).

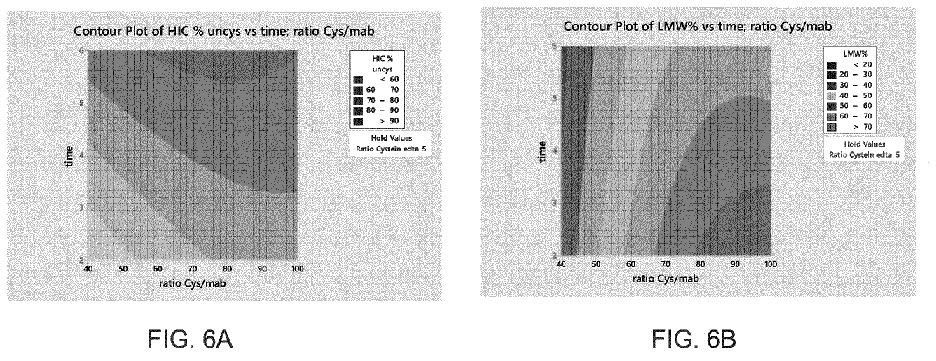

FIG. 6 shows contour plots illustrating the effect of the ratio Cys:mAb and the time on the de-cysteinylation of the antibody and the creation of fragments (LMW). FIG. 6A shows a contour plot of % un-cysteinylated (as measured by HIC-HPLC). The model indicated that a percentage of un-cysteinylated species equal or higher than 70% is reached for a static incubation duration (time) above 5 hours for ratios cysteine:mAb<50 mol/mol whereas the LMW % stays lower than 40% for this ratio and time values.

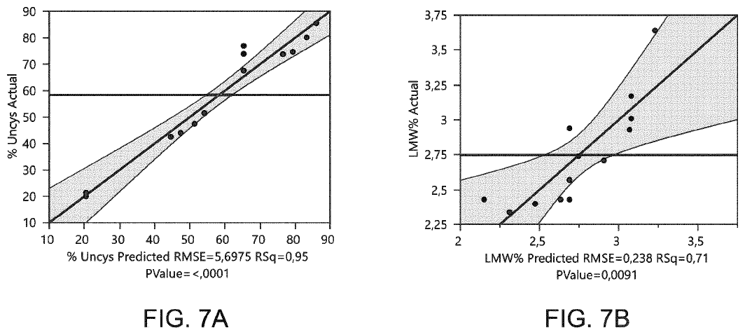

FIG. 7 shows actual vs predicted plots for evaluated responses derived from DOE2 results. FIG. 7A shows a plot for % un-cysteinylated species; FIG. 7B shows a plot for LMW % species. For the un-cysteinylated species percentage, the R² of the statistical model was 0.95, demonstrating a good model fit. For the LMW % response, the R² was 0.71, meaning that 29% of the variability was not explained by the model.

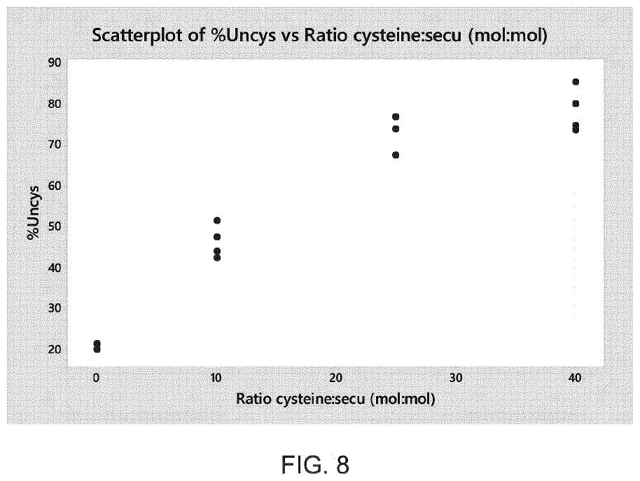

FIG. 8 shows a scatterplot of % un-cysteinylated species vs. the ratio cysteine:antibody of all the runs performed with DOE1 and DOE2. These data show that to reach more than 70% of un-cysteinylated species in the eluate, the ratio Cys:mAb may be set to at least 25 mol/mol.

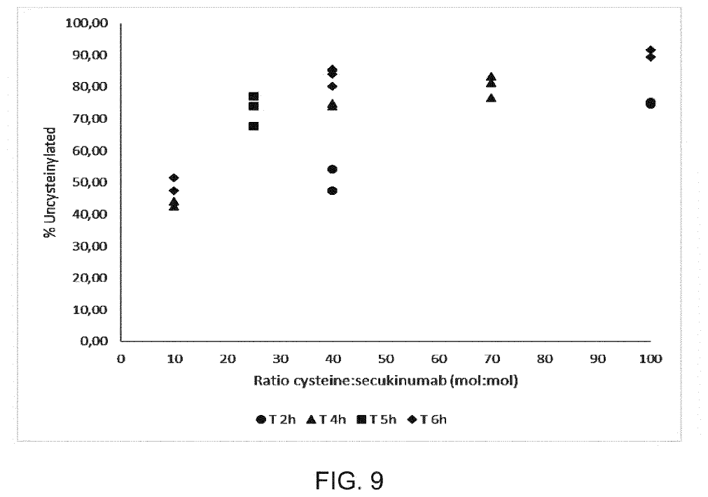

FIG. 9 shows a scatterplot of the evolution of de-cysteinylation percentage (as measured by HIC-HPLC) against the ratio cysteine:antibody in several chromatography runs from DOE1 and DOE 2. FIG. 9 combines the results shown in DOE1 and DOE2 for the un-cysteinylated species. Increasing the ratio of cysteine:antibody results in an increase of the final percentage of un-cysteinylated species from between 40-50% when using a ratio Cys:mAb of 10 to between 70-90% when using a ratio Cys:mAb of 100, depending upon the static incubation time. Static incubation duration (time) also increases this percentage.

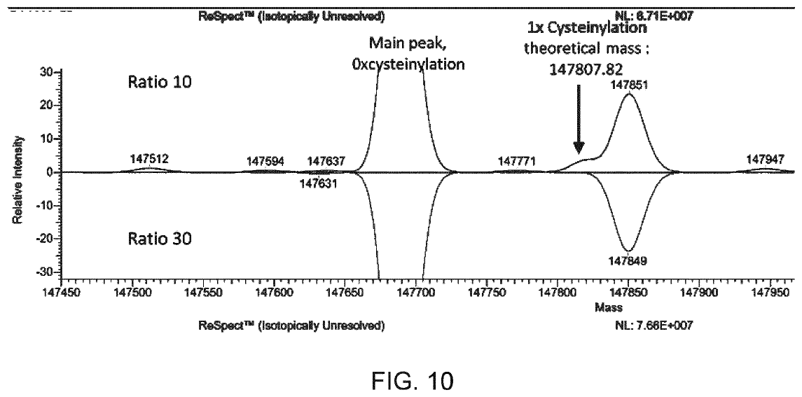

FIG. 10 shows a mirror plot of LC-MS analysis showing the sample representing a 10:1 ratio Cys:mAb on-column treatment on the top panel and 30:1 on the ratio Cys:mAb on-column treatment on the bottom panel (zoomed view 0-30%). The figure shows the antibodies are fully decysteinylated when using a molar ratio of cysteine:antibody of 30:1.

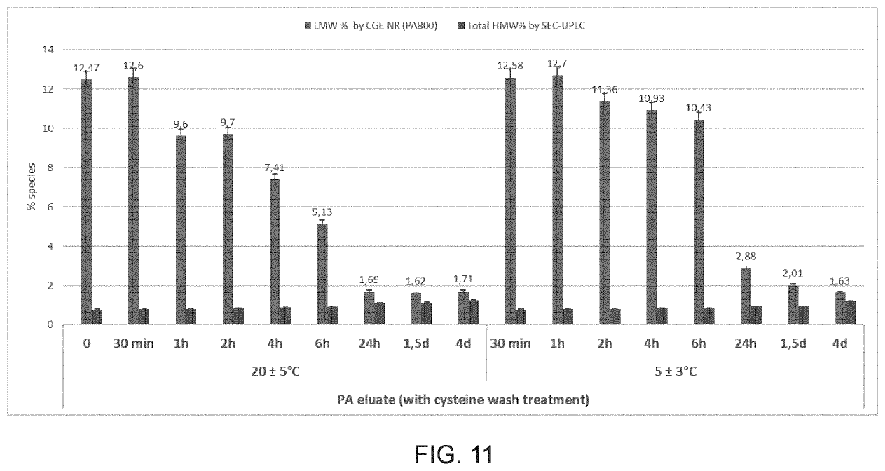

FIG. 11 shows a bar chart representation of the HMW % and LMW % evolution during protein A eluate storage at 20±5° C. and 5±3° C. for 4 days.

Sequence listing

SEQ ID NO: 1 Secukinumab Heavy Chain (full length)
EVQLVESGGGLVQPGGSLRLSCAASGFTFSNYWMNWVRQAPGKGLEWVAAINQDGSEKYYVGSVKGRFTI
SRDNAKNSLYLQMNSLRVEDTAVYYCVRDYYDILTDYYIHYWYFDLWGRGTLVTVSSASTKGPSVFPLAP
SSKSTSGGTAALGCLVKDYFPEPVTVSWNSGALTSGVHTEPAVLQSSGLYSLSSVVTVPSSSLGTQTYIC
NVNHKPSNTKVDKRVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK
SEQ ID NO: 2 Secukinumab Light Chain (full length)
EIVLTQSPGTLSLSPGERATLSCRASQSVSSSYLAWYQQKPGQAPRLLIYGASSRATGIPDRFSGSGSGT
DFTLTISRLEPEDFAVYYCQQYGSSPCTFGQGTRLEIKRTVAAPSVFIFPPSDEQLKSGTASVVCLLNNE
YPREAKVQWKVDNALQSGNSQESVTEQDSKDSTYSLSSTLTLSKADYEKHKVYACEVTHQGLSSPVTKSF
NRGEC
SEQ ID NO: 3 Secukinumab Heavy Chain (variable)
EVQLVESGGGLVQPGGSLRLSCAASGFTFSNYWMNWVRQAPGKGLEWVAAINQDGSEKYYVGSVKGRFTI
SRDNAKNSLYLQMNSLRVEDTAVYYCVRDYYDILTDYYIHYWYFDLWGRG
SEQ ID NO: 4 Secukinumab Light Chain (variable)
EIVLTQSPGTLSLSPGERATLSCRASQSVSSSYLAWYQQKPGQAPRLLIYGASSRATGIPDRESGSGSGT
DFTLTISRLEPEDFAVYYCQQYGSSPCTFGQGTRLEIKRTVAA
SEQ ID NO: 5 Secukinumab Heavy Chain CDR1
NYWMN
SEQ ID NO: 6 Secukinumab Heavy Chain CDR2
AINQDGSEKYYVGSVKGRE
SEQ ID NO: 7 Secukinumab Heavy Chain CDR3
DYYDILTDYYIHYWYFD
SEQ ID NO: 8 Secukinumab Light Chain CDR1
RASQSVSSSYLA
SEQ ID NO: 9 Secukinumab Light Chain CDR2
GASSRAT
SEQ ID NO: 10 Secukinumab Light Chain CDR3
QQYGSSPCTF

The practice of embodiments of the present invention employs, unless otherwise indicated, conventional techniques of molecular biology, microbiology, cell biology, cell culture, biochemistry, and immunology, which are within the skill of those working in the art.

Such techniques are explained fully in the literature, such as, for example, Sambrook et al, Molecular Cloning, A Laboratory Manual (2001) Cold Harbor-Laboratory Press, Cold Spring Harbor, N.Y, Ausubel et al., Current Protocols in Molecular Biology (1990) published by John Wiley and Sons, N.Y, “Animal Cell Culture” (R. I Freshney, ed. 1987) or the like.

Unless defined otherwise, all technical and scientific terms used herein have the same meaning as commonly understood by one of ordinary skill in the art to which this disclosure belongs. For example, the Concise Dictionary of Biomedicine and Molecular Biology, Juo, Pei-Show, 2^{nd} ed., 2002, CRC Press; The Dictionary of Cell and Molecular Biology, 3^{rd} ed., Academic Press; and the Oxford University Press, provide a person skilled in the art with a general dictionary of many of the terms used in this disclosure. For chemical terms, the skilled person may refer to the International Union of Pure and Applied Chemistry (IUPAC).

Units, prefixes and symbols are denoted in their Système International d'Unités (SI) accepted form. Numeric ranges are inclusive of the numbers defining the range.

It is to be noted that the term “a” or “an” entity refers to one or more of that entity; for example, “an antibody” or “a cysteine” is understood to represent one or more antibody or cysteine molecules, respectively. As such, the terms “a” (or “an”), “one or more,” and “at least one” can be used interchangeably herein.

The present methods include the use of antibodies, or antigen-binding fragments, variants, or derivatives thereof. Unless specifically referring to full-sized antibodies such as naturally occurring antibodies, the term “antibodies” encompasses full-sized antibodies as well as antigen-binding fragments, variants, analogs, or derivatives of such antibodies, e.g., naturally occurring antibody or immunoglobulin molecules or engineered antibody molecules or fragments that bind antigen in a manner similar to antibody molecules.

Selective Reduction of Antibodies

In certain embodiments, the invention provides a method of selectively reducing one or more unpaired cysteines of a monoclonal antibody. Typically, the method comprises selectively reducing one or more cysteines in one or more complementary determining region(s) of a monoclonal antibody.

The method of selectively reducing the unpaired cysteine(s) of the monoclonal antibody may be performed during upstream processes of cell culture as further described herein (e.g., during production of the antibody). In addition or alternatively, the method of selectively reducing the unpaired cysteine(s) of the monoclonal antibody may be performed during downstream processes as further described herein (e.g., during “on-column” reduction or other steps of purifying the antibody).

As used herein, the term “selectively reducing” refers to unblocking (e.g., un-cysteinylation and/or un-glutathionylation) of one or more unpaired cysteines, whilst keeping conserved inter and intra-molecular disulfide bonds elsewhere in the antibody intact. For example, unpaired cysteine residues in the CDRs may be reduced whilst no (or only non-significant) reduction may occur elsewhere in conserved cysteine residues in the framework regions of the variable region and/or constant region of the antibody.

As used herein, “non-significant” reduction refers to any transient and/or minor reduction of cysteines engaged in conserved cysteine-cysteine disulfide bonds. However, the majority of these cysteines will re-oxidize to form conserved inter-chain, hinge region and/or intra-chain disulfide bridges.

An ‘unpaired cysteine’ refers to a cysteine that is not involved in conserved antibody disulfide bonding. The status of an unpaired cysteine may be free or non-bonded with any other molecule (e.g., un-cysteinylated and/or un-glutathionylated). Alternatively, the status of an unpaired cysteine may be non-free or bonded with another molecule (e.g., cysteinylated, glutathionylated, reacted with any other component and/or oxidized to sulfinic or sulfonic acid).

Typically, the monoclonal antibodies are recombinantly produced by mammalian cells. For example, the antibodies may have been recombinantly produced by Chinese hamster ovary (CHO) cells, murine myeloma cells (NSO) or the like. The antibodies may be human or humanized. Typically, the antibodies are recombinantly produced by CHO cells. Typically, the antibodies are human. Typically, the antibodies are IgG antibodies, e.g. IgG1 isotype.

Any antibody containing one or more unpaired cysteines may be used in the methods of the invention. Typically, the antibodies that are used contain one or more cysteine(s) in one or more CDRs. Typically, the antibody requires the unpaired cysteine to be free (i.e., un-cysteinylated) for correct structure and/or function (e.g., to recognize antigen). Typically, the cysteine is surface accessible, e.g., not sterically protected from disulfide bond formation as part of a folded region of the antibody. Typically, the cysteine is naturally occurring, e.g., the antibody has not been artificially engineered with any additional cysteine residue to facilitate attachment of any other molecule.

In certain embodiments, the cysteine is in the light chain CDR1, CDR2 or CDR3 or the heavy chain CDR1, CDR2 or CDR3 of the antibody. A typical antibody molecule has two antigen receptors and therefore contains twelve CDRs in total. As such, the antibody may contain two unpaired cysteines if one of the six CDRs has an unpaired cysteine. In this scenario, the disclosed methods are capable of selective reducing both unpaired cysteines.

In preferred embodiments, the antibodies are anti-IL-17 (i.e., IL-17A) antibodies. As used herein, “anti-IL-17 antibodies” include any antibodies (or antigen-binding fragments thereof) capable of specifically binding to IL-17.

In certain embodiments, the anti-IL17 antibody comprises:
(a) a full-length heavy chain having at least about 85%, 90%, 95% or more sequence identity to the amino acid sequence of SEQ ID NO: 1; and/or
(b) a full-length light chain having at least about 85%, 90%, 95% or more sequence identity to the amino acid sequence of SEQ ID NO: 2.

In certain embodiments, the anti-IL17 antibody comprises:
(a) a full-length heavy light chain comprising SEQ ID NO:1; and/or
(b) a full-length light chain comprising SEQ ID NO:2.

In certain embodiments, the anti-IL17 antibody comprises:
(a) a VH sequence having at least about 85%, 90%, 95% or more sequence identity to the amino acid sequence of SEQ ID NO: 3; and/or
(b) a VL sequence having at least about 85%, 90%, 95% or more sequence identity to the amino acid sequence of SEQ ID NO: 4.

As used herein, “sequence identity” refers to a sequence having the specified percentage of amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid sequences. Suitable programs to determine percent sequence identity include for example the BLAST suite of programs available from the U.S. Government's National Center for Biotechnology Information BLAST web site.

In certain embodiments, the anti-IL17 antibody comprises:
(a) a VH sequence comprising SEQ ID NO:3; and/or
(b) a VL sequence comprising SEQ ID NO:4.

In certain embodiments, the anti-IL117 antibody comprises the following CDRs:
(a) HCDR1 comprising the amino acid sequence of SEQ ID NO: 5;
(b) HCDR2 comprising the amino acid sequence of SEQ ID NO: 6;
(c) HCDR3 comprising the amino acid sequence of SEQ ID NO: 7;
(d) LCDR1 comprising the amino acid sequence of SEQ ID NO: 8;
(e) LCDR2 comprising the amino acid sequence of SEQ ID NO: 9; and
(f) LCDR3 comprising the amino acid sequence of SEQ ID NO: 10.

As used herein, the term “Complementarity Determining Regions” (CDRs) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e., about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)). Further information regarding CDR sequences of specific antibodies is available, for example, from the IMGT® database for therapeutic monoclonal antibodies, (Poiron C., Wu Y., Ginestoux C., Ehrenmann, Duroux P. and Lefranc M P. JOBIM 2010, Paper 13 (2010).

In certain embodiments, the invention provides a method for selectively reducing cysteine in the light chain CDR3 of an anti-IL-17 antibody. For example, the light chain CDR3 may have one unpaired cysteine. In this scenario, the disclosed method is capable of reducing this cysteine in both light chains of the anti-IL17 antibody.

In preferred embodiments, the anti-IL-17 antibody is secukinumab, biosimilar or variant thereof as described herein.

In preferred embodiments, the invention provides a method of selectively reducing the cysteine at position 97 of the light chain (LC) of secukinumab. The invention also encompasses methods of selectively reducing the equivalent unpaired cysteine(s) in variants of secukinumab. For example, an “equivalent” unpaired cysteine may be located at a different position of secukinumab, e.g., due to deletions and/or substitutions between secukinumab and the variant antibody.

References herein to “secukinumab” include the originator drug substance (as commercially available), secukinumab as defined in WO2006/013107 A1 (Novartis) (particularly AlN457 therein) and elsewhere in the art, and also biosimilars thereof.

As used herein, “biosimilar” refers to an antibody that is similar to an approved reference antibody (e.g., secukinumab) based upon data derived from (a) analytical studies that demonstrate that the antibody is highly similar to the reference antibody notwithstanding minor differences in clinically inactive components; (b) animal studies (including the assessment of toxicity); and/or (c) clinical studies (including the assessment of immunogenicity and pharmacokinetics or pharmacodynamics) that are sufficient to demonstrate safety, purity, and potency in one or more appropriate conditions of use for which the reference antibody is licensed and intended to be used.

In certain embodiments, the biosimilar and reference antibody (e.g., secukinumab) utilize the same mechanism(s) of action for the condition(s) of use prescribed, recommended, or suggested in the proposed labeling, but only to the extent the mechanism(s) of action are known for the reference antibody. Typically, the condition(s) of use prescribed, recommended, or suggested in the labeling proposed for the biosimilar have been previously approved for the reference antibody. Typically, the route of administration, the dosage form, and/or the strength of the biosimilar antibody are the same as those of the reference product.

In certain embodiments, the facility in which the biosimilar is manufactured, processed, packed, or held meets standards designed to assure that the antibody continues to be safe, pure, and potent. The reference antibody may be approved in at least one of the U.S., Europe, or Japan.

Selective Reduction During Antibody Production

In certain embodiments, the method comprises selectively reducing one or more unpaired cysteines of a recombinant monoclonal antibody during production of the antibody (i.e., during upstream processes of cell culture).

In certain embodiments, the method for selectively reducing the antibody comprises:
(a) providing a cell capable of recombinant expression of the antibody;
(b) culturing the cell in a cell culture medium, wherein the cells are cultured at a first temperature and then shifted to a second temperature, wherein the second temperature is lower than the first temperature,
wherein the cells are maintained in culture until at least 90% or more of the unpaired cysteines are de-cysteinylated; and
(c) harvesting the antibodies from the cell culture to obtain a preparation of the antibody.

In certain embodiments, more than about 90%, 91%, 92%, 93%, 94%, 95%, 96%, 97%, 98% or more of the unpaired cysteines of the antibodies in the harvested sample are free (e.g., un-cysteinylated, un-glutathionylated and/or have the non-oxidized cysteine side chain). In other words, about 10%, 9%, 8%, 7%, 6%, 5%, 4%, 3%, 2%, 1% or less of the antibodies may be blocked (e.g., cysteinylated, glutathionylated and/or reacted with other components via a disulfide link). The cysteinylation of one or more CDRs of an antibody may be determined, for example, by mass-spectrometry or the like.

In certain embodiments, the antibodies are produced by cells comprising a nucleic acid(s) encoding the antibody. In certain embodiments, the antibodies are produced by cells comprising a nucleic acid(s) encoding any antibody as described herein. Typically, the nucleic acid(s) is a recombinant nucleic acid(s). Typically, the antibody is secreted and is released by the cell into the cell culture medium. Typically, the cell is a mammalian cell (e.g., CHO cell) as further described herein.

A nucleic acid encoding the antibody may be introduced into the cell using a wide variety of methods known in the art, including for example, transfection (e.g., lipofection), transduction (e.g., lentivirus, adenovirus, or retrovirus infection), and electroporation. In certain embodiments, the nucleic acid(s) that encodes the antibody may not stably integrate into a chromosome of the mammalian cell (transient transfection). In alternative embodiments, the nucleic acid(s) may stably integrate into a chromosome of the mammalian cell.

In certain embodiments, the nucleic acid(s) encoding the antibody can be present in a plasmid and/or in a mammalian artificial chromosome (e.g., a human artificial chromosome). In certain embodiments, the nucleic acid(s) can be introduced into the cell using a viral vector (e.g., a lentivirus, retrovirus, or adenovirus vector). The nucleic acid(s) may be operably linked to a promoter sequence (e.g., a strong promoter, such as a β-actin promoter and CMV promoter, or an inducible promoter). A vector comprising the nucleic acid(s) may also comprise a selectable marker (e.g., a gene that confers hygromycin, puromycin, or neomycin resistance to the mammalian cell).

The cell capable of recombinant expression of the antibody may be cultured under any suitable conditions that allow production of the antibody. Typically, the cell is cultured under conditions that allow production of the antibody at commercial scale (e.g., 0.5 g/L, 1 g/L, 2 g/L, 3 g/L, 4 g/L, 5 g/L, 6 g/L, 7 g/L, 8 g/L, 10 g/L, 20 g/L or more).

As used herein, the terms “culture”, “culturing”, “cell culture” or “cell culturing” refer to the maintenance or proliferation of a cell under a controlled set of physical conditions. For example, the oxygen levels, temperature, pH and/or duration of the cell culture may be monitored and adjusted to maintain or control cell viability, productivity, % de-cysteinylation, % glutathionylation, % acidic species or the like.

The term “monitored” or “monitoring” refers to the ability to measure specific process parameters or process outputs such as product quality attributes (including % cysteinylation of the antibodies), pH, dissolved oxygen, media components, unit operations and/or flow rate. Monitoring can be applied according to the design of the process to produce the antibody. For example, monitoring can be applied at one or more specific points in the process, for certain steps or time periods within the process, or for the duration of the process. In certain embodiments, the pH of the cell culture is maintained for certain steps or time periods within the process and/or the duration of the process.

In certain embodiments, the pH of the cell culture that is maintained may depend on the cell-line, antibody, cell media, process control strategy and/or other conditions selected for the particular method. The pH during cell culture is typically monitored during cell culture and actively adjusted so it remains constant. For example, the pH may otherwise typically decrease during cell culture. Typically, pH may be regulated through addition of concentrated bases or acids and/or carbon dioxide gassing regulation loop(s).

In certain embodiments, the cells are cultured under a pH of about 6.2 to about 7.6. In certain embodiments, the cells are cultured under a pH of about 6.4 to about 7.4. Typically, the cells are cultured under a pH of about 6.7 to about 7.1. Preferably, the cells are cultured under a pH of about 6.8 to 7.1. More preferably, the cells are cultured under a pH of about 6.95. In certain embodiments, the constant pH comprises defining a pH set point and a pH dead band range. In certain embodiments, the pH set point is between about 6.7 to about 7.1 (e.g., pH 6.7, 6.8, 6.9, 7.0 or 7.1). Preferably, the pH setpoint is 6.95.

The term “pH dead band range” refers to a range through which pH may be varied without initiating a response of adjusting the pH back to the setpoint and/or back to within the dead band range. Accordingly, movement of the pH outside the dead band range results in a response of adjusting the pH back to the setpoint and/or back to within the dead band range. The pH dead band range therefore defines, with regard for the pH set point, an upper and lower pH limit.

In certain embodiments, the pH dead band range comprises a range of between about ±0.1 pH units to about ±0.4 pH units (e.g., ±0.1 pH units, ±0.2 pH units, ±0.3 pH units, ±0.4 pH units). In preferred embodiments, the pH dead band range is about ±0.15 pH units.

In certain embodiments, the cells are maintained in culture until at least 90% or more of the unpaired cysteines are de-cysteinylated. For example, % de-cysteinylation may be monitored one or more times during production of the antibody using any suitable technique. Once at least about 90% de-cysteinylated is achieved, the cells may be harvested from the cell culture to obtain a preparation of the antibody.

In certain embodiments, any active monitoring of the % de-cysteinylation is not performed during antibody production. For example, depending on the specific process, the skilled person could determine the total duration of cell culture required to achieve a desired % de-cysteinylation of the antibodies during one or more test runs. During commercial production, the antibodies could then be cultured for this set duration without requiring to monitor % de-cysteinylation of the antibodies.

The duration of cell culture may depend, for example, on the cell-line, antibody, cell media, process control strategy and/or other conditions selected for the method.

In certain embodiments, the duration of cell culture may be at least about 1, 2, 3, 4, 5, 6, 7, 8, 9, 10, 11, 12, 13, 14, 15, 16 or 17 days. Typically, the duration of cell culture is between about 14 to about 17 days.

In certain embodiments, the temperatures of the cell culture may depend on the cell-line, antibody, cell media, process control strategy and/or other conditions selected for the method. For example, different antibodies may have different stabilities at certain temperatures.

In certain embodiments, the cells are cultured at a first temperature and then actively shifted to a second temperature. For example, the cells are cultured at a first temperature of about 300 to about 40° C. (e.g., 31° C., 32° C., 33° C., 34° C., 35° C., 36° C., 37° C., 38° C., or 39° C.). Typically, the second temperature is about 3° C. to about 5° C. lower than the first temperature (e.g., about 4° C. lower). In preferred embodiments, the cells may be cultured at a first temperature of about 37° C. and a second temperature of about 33° C.

The temperature may be lowered at any stage of cell culture. Typically, the temperature is lowered during day 10, 11 or 12 of cell culture. In preferred embodiments, the temperature may be lowered during day 10 of the culture. Typically, the temperature may be lowered from any first temperature as described herein to any second temperature as described herein.

In certain embodiments, the antibodies are produced by mammalian cells. In certain embodiments, the mammalian cells can be a cell that grows in suspension or an adherent cell. In certain embodiments, the mammalian cells may be Chinese hamster ovary (CHO) cells (e.g., CHO DG44 cells or CHO-K1 s cells), Sp2.0, myeloma cells (e.g., NS/0), B-cells, hybridoma cells, T-cells, human embryonic kidney (HEK) cells (e.g., HEK 293E and HEK 293F), African green monkey kidney epithelial cells (Vero) cells, or Madin-Darby Canine (Cocker Spaniel) kidney epithelial cells (MDCK) cells. In some examples where an adherent cell is cultured, the culture can also contain a plurality of microcarriers (e.g., microcarriers that contain one or more pores). Additional mammalian cells that can be cultured in any of the processes described herein are known in the art.

In preferred embodiments, the mammalian cell is a CHO cell.

In certain embodiments, the antibody is a secreted by the mammalian cell into the culture medium. For example, a nucleic acid sequence encoding the antibody can contain a sequence that encodes a secretion signal peptide at the N- or C-terminus of the antibody, which is cleaved by an enzyme present in the mammalian cell, and subsequently released into the culture medium.

Typically, the cells may be cultured in a bioreactor, holding tank, or a non-bioreactor unit operation vessel. In certain embodiments, unclarified harvest may be obtained from such bioreactor processes as fed-batch, batch, or perfusion (continuous) processes. In certain embodiments, the methods can be performed in a vessel separate from the bioreactor. For example, the methods may be performed within a separate reactor designed to achieve selective reduction of the antibodies. In preferred embodiments, the cells are cultured in a bioreactor.

In certain embodiments, the bioreactor may have a volume of, e.g., between about 1 L to about 10,000 L or more.

In certain embodiments, the bioreactor holding tank, or a non-bioreactor unit operation vessel may also be used to cool the culture (e.g., at a temperature of less than about 25° C., less than about 15° C., or less than about 10° C.) or heat the culture (e.g. at temperature greater than about 25° C., greater than about 30° C. or greater than about 35° C. (e.g., 37° C.).

The term “fed-batch process” or “fed-batch culture” refers to a culturing process wherein the culturing of the cells comprises the periodic or continuous addition of a further liquid culture medium and/or cell feed to the first liquid culture medium without substantial or significant removal of the first liquid culture medium or further liquid culture medium from the cell culture. The further liquid culture medium and/or cell feed may be the same as the first cell culture medium. Alternatively, the further liquid culture medium and/or cell feed may not be the same as the first cell culture medium.

The term “batch culture” or “batch process”, refers to a culturing process wherein the culturing of the cells comprises initial inoculation of the cells into a fresh medium and no further nutrient is added until the target product is produced.

The term “perfusion culture, “perfusion process”, “continuous culture” or “continuous process” refer to a culturing process wherein the culturing of the cells comprises the periodic or continuous removal of a first liquid culture medium and at the same time or shortly thereafter adding substantially the same volume of a second liquid cell culture and/or cell feed to the culture.

In certain embodiments, the cells may be cultured in a batch bioreactor. In certain embodiments, the cells may be cultured in a fed-batch bioreactor. Culturing a cell in a fed-batch bioreactor comprises the addition to a first culture medium a supplemental cell feed and/or a further cell culture medium. The addition may be periodic or continuous addition of a supplemental cell feed and/or a further cell culture medium.

In certain embodiments, the cells may be cultured in a perfusion bioreactor. Culturing a cell in a perfusion (continuous) bioreactor comprises the periodic or continuous removal of a first liquid culture medium and at the same time or shortly thereafter adding substantially the same volume of a second liquid cell culture and/or cell feed to the culture.

In certain embodiments, the cells are cultured in one or more cell culture medium. Typically, the cell culture medium is a liquid medium. In certain embodiments, the liquid culture media can be supplemented with a mammalian serum (e.g., fetal calf serum and bovine serum), and/or a growth hormone or growth factor (e.g., insulin, transferrin, and epidermal growth factor). Alternatively, the liquid culture media (e.g., a first and/or further liquid culture medium) can be a chemically defined liquid culture medium, an animal-derived component free liquid culture medium, a serum-free liquid culture medium, or a serum-containing liquid culture medium. Such media are widely available on the market, and any suitable medium may be used in the methods described herein.

In certain embodiments, commercially available media such as Minimal Essential Medium ((MEM), Sigma), RPMI-1640 (Sigma), and Dulbecco's Modified Eagle's Medium ((DMEM, Sigma) are suitable for culturing the host cells. Typically, the cell media is Excell Advanced Medium as also commercially available.

The culture medium can be supplemented with desired substrates (e.g., a mammalian hormone or growth factor (e.g., insulin, transferrin, or epidermal growth factor), salts and buffers (e.g., calcium, magnesium, and phosphate, nucleosides and bases protein, tissue hydrolysates, etc.) In certain embodiments, the cell culture medium is supplemented with a mannosidase I inhibitor. Mannosidase inhibitors are effective for increasing high mannose N-linked glycosylation in monoclonal antibody production. N-linked glycosylation is an important attribute for drug safety and efficacy. In certain embodiments, the mannosidase I inhibitor is Kifunensine. In certain embodiments, the Kifunensine is present in the cell culture medium at a concentration of less than about 5 μg/kg, e.g., about 4 μg/kg, about 3 μg/kg, about 2 μg/kg, about 1 μg/kg or less.

In certain embodiments, a supplemental cell feed and/or a further cell culture medium may be added to the first cell culture medium. For example, the cell culture medium may be supplemented with cell feed and/or further cell culture medium each day of the cell culture from about day 2, 3, 4, 5, 6, 7, 8, 9, 10, or later to the penultimate day or the last day of the cell culture. In certain embodiments, the addition of the supplemental cell feed and/or further cell culture medium may depend on the cell-line, antibody, cell media, process control strategy and/or other conditions selected for the particular method. In preferred embodiments, the cell culture medium is supplemented with cell feed and/or further cell culture medium each day from about day 2, 3 or 4 of the culture to the penultimate day of the culture.

In certain embodiments, supplemental glucose may be added to the cell culture medium. For example, the supplemental glucose may be added to the cell culture medium to a concentration of between about 2 g/L to about 7, g/L, e.g., about 2 g/L, about 3 g/L, about 4 g/L, about 5 g/L about 6 g/L or about 7 g/L. In preferred embodiments, supplemental glucose is added to the cell culture medium to a concentration of about 6 g/L. In certain embodiments, the supplemental glucose is added to the cell culture medium when the concentration of glucose in the cell culture medium falls below about 2 g/L, about 3 g/L or about 4 g/L. In certain embodiments, the addition of the supplemental glucose may depend on the cell-line, antibody, cell media, process control strategy and/or other conditions selected for the method.

In certain embodiments, the cell culture medium may be stirred or agitated, e.g., constantly, or intermittently, using any means of stirring or agitating. In certain embodiments, stirring may be axial or radial. In certain embodiments, agitation may be induced by rocking, rotation, or wave-induced agitation. In certain embodiments, the cell culture is constantly stirred or agitated at rate of between about 60-200 rpm (e.g., about 50 rpm, about 60 rpm, about 70 rpm, about 80 rpm, about 90 rpm, about 100 rpm, about 110 rpm, about 120 rpm, about 130 rpm, about 140 rpm, about 150 rpm, about 160 rpm, about 170 rpm, about 180 rpm, about 190 rpm, or 200 rpm). In preferred embodiments, the cell culture is stirred or agitated at a rate of about 160 to about 180 rpm (e.g., 170 rpm).

In certain embodiments, the cell culture may be stirred at a controlled tip speed. The skilled person would understand that appropriate agitation-related shear forces (e.g., spin rates or impeller tip speed) may be selected, for example, on the scale of the culture.

In certain embodiments, gas may be added to the cell culture. Any suitable gas may be used, including, for example, oxygen (O₂), carbon dioxide (CO₂), nitrogen (N₂), and different compositions of mixed gasses, such as 20% O₂/10% CO₂/70% air or 20% O₂/5% CO₂/75% air. Any dissolved gas level may be used during cell culture. Once selected, the gas concentration may be optimized according to the cell-line, antibody, cell media, process control strategy and/or other conditions being used.

In certain embodiments, the cell culture may comprise about 20% to about 50% dissolved oxygen (DO), typically about 30% to about 40%. Typically, a DO cascade is applied to the cell culture. For example, an automatic two gas mix cascade of Air/O2 may be used. Preferably, the DO cascade includes O₂ enrichment. The cascade or other parameters that are selected may typically depend on the desired % DO output.

The term “dissolved oxygen” or “DO” refer to the amount of oxygen that is dissolved in a given medium. It can be measured with an oxygen probe using methods established in the art. Percent oxygen saturation is the amount of oxygen in a solution relative to the total amount of oxygen that the solution can hold at a particular temperature. The levels of dissolved oxygen may be regulated e.g., through gassing (sparge and/or in overlay) of multiple gasses (air, oxygen, carbon dioxide, nitrogen) and/or agitation regulation loop(s). In certain embodiments, the cell culture comprises inoculating the cell culture medium with a cell capable of recombinant expression of an antibody of interest. In certain embodiments, the cell culture comprises inoculating the cell culture medium at a seeding density of between about 0.2×10⁶ cells/ml to about 0.6×10⁶ cells/ml (e.g., about 0.2×10⁶ cells/ml, about 0.3×10⁶ cells/ml, 0.4×10⁶ cells/ml, about 0.5×10⁶ cells/ml, or 0.6×10⁶ cells/ml. In preferred embodiments, the seeding cell density is about 0.4×10⁶ cells/ml. In certain embodiments, the target seeding density may be adjusted depending on the cell-line, antibody, cell media, process control strategy and/or other conditions selected for the method. For example, the target seeding density may be about 0.4×10⁶ cells per ml. In certain embodiments, the cells are maintained in culture until a designated viable cell density is reached. For example, the viable cell density may be at least about 2.0×10⁶ cells per ml.

The term “target seeding density” refers to the cell density within the cell culture medium at the initiation of the culture.

In certain embodiments, the cell culture medium may further comprise an antifoam emulsion. Antifoam emulsion acts to eliminate excessive foaming formed during cell disruption, due to the presence of proteins, lipids and/or carbohydrates in the culture medium. Production of foam is often undesirable and can cause defects on surface coatings and prevent the efficient filling of containers. In certain embodiments, the antifoam emulsion may be a silicone antifoam emulsion. Antifoam emulsions, including silicone antifoam emulsions are commercially available.

The cells produced by the cell culture processes described herein may be harvested to provide a preparation of antibodies. In certain embodiments, the cells may be harvested from the cell culture by centrifugation, flocculation, depth filtration and/or tangential flow filtration. Such techniques are well established in the art. The term “harvested” refers to the separation of the antibody from the cells and cell debris of the cell culture.

In certain embodiments, the harvested antibodies (clarified cell culture fluid) is subjected to any one or more of the additional downstream processing steps as described herein.

In certain embodiments, the method comprises passing the harvested antibodies through one or more further chromatography materials (see ‘further purification of antibodies’)

Binding of Antibodies to Chromatography Material

In certain embodiments, the method of selectively reducing one or more unpaired cysteines comprises a step of contacting a sample of the monoclonal antibodies with one or more chromatography material(s). Typically, the sample is passed through the chromatography material one or more times thereby adsorbing the antibodies to the chromatography material. As described herein, any sample containing antibodies having at least one or more unpaired cysteine(s) that is amenable to application to chromatographic material may be used. Typically, the sample is isolated from any cell culture process as described herein.

In certain embodiments, the sample of antibodies is harvested from mammalian (e.g., CHO) cells. For example, the sample may be obtained by centrifugation of harvested mammalian cell culture (with or without subsequent clarification). Typically, the sample is clarified cell culture fluid. Typically, the clarified cell culture fluid is obtained from a CHO cell culture bioreactor.

In certain embodiments, the sample is obtained from mammalian suspension cell culture using any standard techniques in the art. For example, during upstream processes the levels of dissolved oxygen may be regulated e.g., through gassing (sparge and/or in overlay) of multiple gasses (air, oxygen, carbon dioxide, nitrogen) and/or agitation regulation loop(s). In addition, pH may be regulated through addition of concentrated bases or acids and/or carbon dioxide gassing regulation loop(s). Commercial upstream processes for recombinantly producing monoclonal antibodies are described in the art.

Typically, the cells are cultured using commercially available cell culture media. Typically, the quantity and/or rate of nutrient feed, amino acid or other nutrient supplements provided to the cell culture may be monitored and/or manipulated during the upstream processes as further discussed above.

In certain embodiments, the sample is obtained following centrifugation, flocculation, depth filtration and/or tangential-flow filtration (TFF) of the cell culture broth. Such techniques are well established in the art. Typically, the sample is a particle-free feed to downstream processes such as Protein A chromatography as further described herein.

In certain embodiments, the sample is stored before use. For example, the sample may be frozen for any period of time, e.g., one week, two weeks, three weeks, one month, two months, three months or more. Typically, the sample is thawed at least about 24 hours or less before use. Alternatively, the sample may be loaded directly onto the chromatography material following upstream processing.

In certain embodiments, about 91%, 92%, 93%, 94%, 95%, 96%, 97%, 98%, 99% or more of the unpaired cysteines of the antibodies in the initial sample for downstream processing are free (e.g., un-cysteinylated, un-glutathionylated and/or have the non-oxidized cysteine side chain). For example, the optimized upstream processes described herein may lead to antibodies having such a high % of de-cysteinylation. Thus, any further selective reduction of the sample during downstream processing may not be required to retain or improve biological activity and/or structure of the antibodies as compared to a reference antibody. In such embodiments, any standard downstream processing of antibodies may be performed as well known in the art.

In alternative embodiments, about 90%, 85%, 80%, 75%, 70%, 65%, 60%, 55%, 50% or less of the unpaired cysteines of the antibodies in the initial sample are free (e.g., un-cysteinylated, un-glutathionylated and/or have the non-oxidized cysteine side chain). In other words, the initial sample used in the methods of the invention may comprise antibodies having a blocked status of unpaired cysteines (e.g., cysteinylated, glutathionylated and/or reacted with other components via a disulfide link) in about 10%, 15%, 20%, 25%, 30%, 40% or 50% or more of the total antibodies. Thus, selective reduction of the sample during subsequent downstream processing steps may be required to retain or improve biological activity and/or structure of the antibodies as compared to a reference antibody.

In certain embodiments, following the methods of downstream selective reduction described herein (e.g., “on-column” reduction), more than about 90%, 91%, 92%, 93%, 94%, 95%, 96%, 97%, 98%, 99% or more of the unpaired cysteines of the antibodies in the sample are free (e.g., un-cysteinylated, un-glutathionylated and/or have the non-oxidized cysteine side chain). In other words, after contacting the antibodies with the mixture comprising the reducing agent (and optionally any additional steps of purifying and/or isolating the antibodies), about 10%, 9%, 8%, 7%, 6%, 5%, 4%, 3%, 2%, 1% or less of the antibodies may be blocked (e.g. cysteinylated, glutathionylated and/or reacted with other components via a disulfide link).

Typically, the downstream processes described herein lead to about a 5%, 10%, 20%, 30%, 40%, 50%, 60% or more increase in un-cysteinylated antibodies as compared to control methods without the step of contacting the antibodies with a mixture comprising one or more reducing agents.

In certain embodiments, the method of the invention may be optimized depending on the initial % of un-cysteinylated antibodies in the sample being loaded onto the chromatography material. For example, the molar ratio of the reducing agent:antibody and/or total duration of contact of the reducing agent with the antibody may be adjusted depending on the % of un-cysteinylated antibodies in the initial sample.

The level of cysteinylation of unpaired cysteines in the antibodies within the sample may be measured by any suitable technique. For example, HIC-HPLC may be used to determine % un-cysteinylated antibodies following standard techniques well described in the art.

In alternative embodiments, the sample may comprise antibodies that have already been subject to one or more downstream processing steps. For example, the sample may be obtained from an eluate of an affinity column (e.g., protein A or the like). In such embodiments, the sample may be undergoing an additional chromatography step during which “on-column” selective reduction may be performed.

In alternative embodiments, the sample may comprise antibodies resulting from additional downstream steps, e.g., cation exchange column, anion exchange column, depth filtration, Polisher technology (e.g., 3 M Polisher ST), nanofiltration, ultrafiltration or the like.

In certain embodiments, the antibodies are contacted with the chromatography material at a loading capacity of about 10%, about 20%, about 30%, about 40%, about 50%, about 60%, about 70%, about 80%, about 90% or more of the dynamic binding capacity (DBC) determined at about 10% breakthrough. As used herein, the term “DBC” refers to the maximal amount of antibody that will bind to the chromatographic material under typical conditions before any significant antibody levels are measured in the flow through (i.e., the breakthrough point). Typically, the antibodies are contacted with the chromatography material (e.g., Protein A) at about 80% of the maximal DBC at about 10% breakthrough.

Any concentration of antibody may be contacted with the chromatography material(s). The concentration of antibody may be adjusted to any preferred concentration prior to contact with the chromatography material, e.g., using water, buffer or the like. Typically, however, the concentration of the antibody in the sample is not adjusted prior to binding with the chromatography material.

In preferred embodiments, the sample is clarified cell culture fluid. Typically, the sample comprises between about 1 to 10 mg/ml antibody, e.g., between about 1.5 and 6.6 mg/ml antibody.

In certain embodiments, about 1 mg to about 100 mg antibody is loaded per ml of chromatography material, e.g., a loading capacity of between about 25 mg/ml to about 75 mg/ml of antibody. Typically, about 50 mg/ml antibody (e.g., secukinumab, biosimilar or variant thereof) is loaded per ml of chromatography material (e.g., Protein A).

The antibodies may be contacted with the chromatography material during loading for any suitable period of time (e.g., any suitable residence time). For example, the antibodies may be contacted with the chromatography material (e.g., Protein A) for between about 2 to about 6 minutes, e.g., about 4 minutes.

Any suitable chromatography material may be used. For example, the chromatography material may be one to which the antibodies in the sample are capable of binding, i.e., a chromatographic material that does not operate in a flow-through mode. Binding of the antibodies to the chromatography material may provide certain advantages, for example, by limiting motion of the antibody and thereby reducing the formation of undesirable antibody complexes during the reduction step.

In certain embodiments, the chromatography material is one or more resin(s). The resin may be packed to a solid support and/or housed within a column. Alternatively, the resin may be within a suspension slurry. Typically, the sample comprising the antibodies is passed through the chromatography column one or more times, thereby binding the antibodies to the chromatography material.

The sample may be passed through the column under any suitable conditions, e.g., at any suitable temperature and/or flow rate. The conditions that are selected may depend on the sample and/or chromatography material being used for the selective reduction step.

In certain embodiments, the chromatography material may comprise a strong cation exchange chromatography resin. For example, the resin may comprise sulphopropyl groups or the like. Alternatively, the chromatography material may comprise a weak cation exchange chromatography resin. For example, the resin may comprise carboxymethyl or the like.

In certain embodiments, the chromatography material may comprise a strong anion exchange chromatography resin. For example, the resin may comprise quaternary ammonium groups or the like. Alternatively, the chromatography material may comprise a weak anion exchange chromatography resin. For example, the resin may comprise diethylaminoethyl, diethylaminopropyl or the like.

In certain embodiments, the chromatography material may comprise an affinity resin. As used herein, “affinity chromatography” is a method that makes use of specific, reversible interactions between the material and the antibody.

In certain embodiments, the resin may be a protein A affinity chromatography resin, protein G affinity chromatography resin, protein A/G affinity chromatography resin, protein L affinity chromatography resin, immobilized metal affinity chromatography resin (e.g., Nickel-NTA resin or Cobalt-NTA resin), Glutathione S-transferase (GST) affinity chromatography resin or the like.

In certain embodiments, the resin is a hydrophobic interaction resin (e.g., butyl resin or octyl resin), or size exclusion chromatography resin. In certain embodiments, the chromatography material may be multi-modal chromatography (MMC) material.

In certain embodiments, the resin is immobilized onto a solid support and/or adjusted to a desired pH as further described herein.

In preferred embodiments, the chromatography material is protein A. Protein A chromatography makes use of the affinity of the IgG binding domains of Protein A for the Fc portion of an immunoglobulin molecule and is well described in the art (Vola et al. Cell Biophys. 24-25: 27-36, 1994; Gagnon, Protein A affinity chromatography, In: Purification tools for monoclonal antibodies, 1996, Validated Biosystems, Tucson, Ariz., 1996; Aybay and Imir, J. Immunol. Methods 233(1-2): 77-81, 2000; Ford et al., J. Chromatogr. B 754: 427-435, 2001; Fahrner et al, Biotechnology and Genetic Engineering News, 18: 301-327, 2001) herein incorporated by reference.

Typically, the Protein A is immobilized on a solid support, e.g., non-aqueous matrix onto which Protein A adheres. Suitable solid supports include, for example, agarose, sepharose, glass, silica, polystyrene, or the like.

In certain embodiments, the Protein A is immobilized on a solid support and equilibrated to a neutral pH within a column. The sample containing the desired antibodies (e.g., clarified cell culture fluid) may then be loaded directly onto the Protein A column. The Protein A column may be washed one or more times before or after loading the antibodies to remove any contaminants.

In certain embodiments, the chromatography material (e.g., Protein A) is pre-washed with an equilibration buffer to prepare for loading with the antibody. Typically, the equilibration buffer is isotonic. Typically, the equilibration buffer is at a pH from about 6 to 8. Typically, at least 5×, 10×, 15×, 20× or more column volumes of the equilibration buffer are washed through the chromatography material. In certain embodiments, the equilibration buffer is a phosphate buffer, Tris buffer, acetate buffer, citrate buffer or the like.

In certain embodiments, the equilibration buffer may include an agent that reduces electrostatic interactions including salts, e.g., sodium salts, potassium salts, ammonium salts, citrate salts, calcium salts, magnesium salts or the like.

In preferred embodiments, the equilibration buffer is a Tris buffer. Typically, the Tris buffer has a pH of about 6 to 8. Typically, the equilibration buffer further comprises one or more salts (e.g., NaCl). By way of example only, the equilibration buffer may comprise about 50 mM Tris-HAc, about 150 mM NaCl and have a pH of about 7.4.

The equilibration buffer may be passed through the chromatography material at any suitable flow rate. The flow rate may depend on the column size. For example, the equilibration buffer may be passed through the chromatography material at a flow rate between about 10 cm/h to about 1000 cm/h, preferably between about 300 cm/h to 400 cm/h (e.g., for large scale applications) or between about 25 cm/h to about 300 cm/h (e.g., for small scale applications). For example, the mixture may be passed through the chromatography material at a flow rate of about 50, 100 or 150 or 200 cm/h. Typically, the flow rate is about 100, 200, 300, 400 or 500 cm/h.

The equilibration buffer may be contacted with the chromatography material and/or antibodies at any suitable temperature. Typically, the temperature is between about 15° C. to 25° C. degrees (e.g., room temperature).

In certain embodiments, the loaded chromatography material may be washed one or more times after the antibodies have been contacted with the chromatography material, to remove any host cell (unbound) contaminants prior to the reduction step. For example, one or more post-loading buffers may be used after the antibodies have been loaded onto the chromatography material.

In certain embodiments, the equilibration buffer is used as a post-loading buffer one or more times after the antibodies have been contacted with the chromatography material, to remove any host cell (unbound) contaminants prior to the reduction step. For example, the loaded chromatography material may be washed with at least 1×, 5×, 10×, 15× or more column volumes with the equilibration buffer. Alternatively, a different post-loading buffer may be used.

In certain embodiments, the loaded chromatography material is washed twice prior to the reduction step (e.g., “wash 1” and “wash 2” as further described herein). Typically, the wash conditions (e.g., column volumes, temperature, flow rate) are similar or the same as the equilibration wash. Typically, the first post-loading buffer is the same as the equilibration buffer. Typically, the second wash after the sample loading is at a higher salt concentration (e.g., about 1 M NaCl) as compared to the equilibration wash buffer (e.g., about 150 mM NaCl).

In certain embodiments, the sample of antibodies is incubated with the chromatography material (e.g., Protein A) under conditions that allow binding of the antibodies to the chromatography material. For example, the antibodies (e.g., secukinumab, variant or biosimilar thereof) may be incubated with protein A resin using any standard conditions described in the art.

Once the antibodies are bound to the chromatography material (and optionally washed as described above), they may subsequently be contacted with a mixture comprising one or more reducing agents as further described herein.

“On-Column” Reduction

In certain embodiments, optional downstream processes of selectively reducing one or more cysteine(s) comprise contacting the bound antibodies with a mixture comprising one or more reducing agents. For example, the method may further comprise passing one or more reducing agent(s) through the chromatography material, wherein the antibodies are contacted with the reducing agent whilst adsorbed to the chromatography material.

As described above, such additional “on-column” reduction may be required in situations where the antibodies in the harvest cell clarified fluid have a low % de-cysteinylated antibodies (e.g., about 90%, 89%, 88%, 87%, 86%, 85%, 84%, 83%, 82%, 81%, 80% or less).

In such embodiments, the invention provides a method of selectively reducing one or more unpaired cysteines of a monoclonal antibody, wherein the method comprises:
(a) contacting a sample of antibodies with chromatography material, thereby binding the antibodies to the chromatography material;
(b) contacting the bound antibodies with a mixture comprising one or more reducing agents; and
(c) eluting the antibodies from the chromatography material.

In certain embodiments, the cysteine is in the complementary determining region (CDR) of the antibody.

In certain embodiments, the antibody is an anti-IL-17 antibody.

In certain embodiments, the antibody comprises:
(a) a VH sequence having at least about 90% or more sequence identity to the amino acid sequence of SEQ ID NO: 3; and
(b) a VL sequence having at least about 90% or more sequence identity to the amino acid sequence of SEQ ID NO: 4.

In certain embodiments, the antibody is secukinumab, biosimilar or variant thereof.

In certain embodiments, the method comprises selectively reducing light chain (LC) Cys97.

In certain embodiments, the chromatography material is Protein A.

In certain embodiments, the sample is clarified cell culture fluid.

In certain embodiments, about 90% or less of the unpaired cysteines of the antibodies in the sample used in step (a) are un-cysteinylated.

In certain embodiments, about 25 mg to about 75 mg of antibody is loaded per mL of chromatography material.

In certain embodiments, the molar ratio of the reducing agent:antibody is:
(i) about 5:1 to about 100:1;
(ii) about 10:1 to about 40:1; or
(iii) about 20:1 to about 30:1.

In certain embodiments, the mixture is contacted with the antibodies for about 5 to about 7 hours.

In certain embodiments, the mixture is at a pH of between about 7.3 to about 8.5.

In certain embodiments, the pH of the mixture is about 8.0.

In certain embodiments, the bound antibodies are contacted with the mixture at a temperature between about 15° C. to about 25° C.

In certain embodiments, the reducing agent is cysteine.

In certain embodiments, the mixture further comprises one or more oxidation reagents.

In certain embodiments, the oxidation reagent is cystine.

In certain embodiments, the molar ratio of reducing agent to cystine is between about 10:1 to about 20:1.

In certain embodiments, the mixture is passed through the chromatography material at a flow rate between about 100 to about 500 cm/h.

In certain embodiments, the flow of the mixture through the chromatography material is paused one or more times, thereby allowing static contact of the mixture with the antibodies bound to the chromatography material, optionally wherein:
(i) the flow of the mixture is paused at least twice;
(iii) the flow of the mixture is paused for about 40, 80 or 120 minutes; and/or
(iii) the static contact of the mixture with the antibodies is between about 3-6 hours in total.

In certain embodiments, the chromatography material is washed with one or more equilibration buffers and/or post-loading buffers prior to step (b) and/or the chromatography material is washed with one or more post-wash buffers after step (b).

In certain embodiments, the equilibrium buffers and/or post-loading buffers are at a pH of about 7.4 and/or the post-wash buffers are at pH of about 5.5.

In certain embodiments, step (c) comprises passing an elution buffer through the chromatography material.

In certain embodiments, the elution buffer is at a pH of about 3.7.

In certain embodiments, the eluted antibodies are neutralized to a pH of about 5.5 to about 7.8.

In certain embodiments, the eluted antibodies are incubated under conditions that decrease the amount of any low molecular weight (LMW) fragments in a sample of the eluted antibodies.

In certain embodiments, the eluted antibodies are incubated at a temperature between about 15° C. to about 25° C.

In certain embodiments, the eluted antibodies are incubated for about 24 hours.

In certain embodiments, the eluted antibodies are incubated in an elution buffer at a neutral pH.

In certain embodiments, the elution buffer is at a pH of about 7.4 to about 7.8.

In certain embodiments, the elution buffer is an acetate buffer.

In certain embodiments, the method further comprises:
(d) passing the eluted antibodies through one or more further chromatography material(s), thereby obtaining a purified preparation of antibodies.

In certain embodiments, the method further comprises:
(d) passing a sample of the eluted antibodies through one or more further chromatography material(s), thereby binding the antibodies to the further chromatography material(s);
(e) contacting the bound antibodies with a wash buffer, wherein the wash buffer is at a pH above about 7.0; and
(f) eluting the antibodies from the further chromatography material(s).

In certain embodiments, the further chromatography material is cation-exchange (CEX) material.

In certain embodiments, about 10 g/L of antibodies or less are loaded onto the further chromatography material(s), and the wash buffer is at a pH of about 8.8 to about 9.0 or more than about 10 g/L of antibodies are loaded onto the further chromatography material(s) and the wash buffer is at a pH of about 7.0 to about 8.4, preferably about 8.0.

In certain embodiments, the wash buffer is a Tris buffer.

In certain embodiments, the further chromatography material(s) are washed with one or more equilibration buffer(s) and/or loading buffer(s) prior to step (e); the bound antibodies are contacted with one or more pre-wash buffers prior to step (e); the bound antibodies are contacted with one or more post-wash buffers after step (e); and/or the bound antibodies are contacted with one or more re-equilibration buffers after step (e).

In certain embodiments, the equilibrium buffer(s), loading buffer(s) and/or re-equilibrium buffer(s) are at a pH of about 5.5; and/or (ii) the pre- and/or post-wash buffers are at pH of about 7.4.

In certain embodiments, step (f) comprises passing an elution buffer through the chromatography material, wherein the elution buffer is at a pH of about 5.5.

As described herein, the mixture comprising the reducing agent(s) may be any solution that is compatible with the chromatography material (e.g., does not disrupt the integrity of the column) and is compatible with the antibodies in the sample (e.g., does not irreversibly denature or inactivate the antibodies).

Any suitable reducing agent may be used in the method of the invention. A suitable reducing agent is also compatible with the chromatography material and antibodies in the sample. Typically, the reducing agent is capable of delivering hydrogen to the one or more unpaired cysteines of the antibody.

In certain embodiments, the reducing agent is a sulfhydryl-group containing reducing agent. In other words, the reducing agent may be a thiol-containing reducing agent (e.g., having an R—SH group).

In certain embodiments, the reducing agent has an oxidation-reduction potential (E°) of about −0.18V to about −0.26V. Typically, the reducing agent has an E° of about −0.20V to about −0.24V preferably about −0.20V to about −0.23V. Techniques for measuring E° are well known in the art, including, for example, thiol-disulfide exchange. The E° values as described herein are typically measured at pH 7, 25° C.

In certain embodiments, the reducing agent is dithiothreitol (DTT), 2-mercaptoethanol, 2-mercaptoacetic acid, cysteine (CSH), cysteamine, glutathione (GSH), tris(2-carboxyethyl)phosphine (TCEP), copper sulphate (CuSO₄) or any combination thereof.

In preferred embodiments, the reducing agent is cysteine. Typically, the mixture comprising the reducing agent also includes cystine. For example, cysteine (CSH) may be spontaneously oxidized into cystine (CSSC) in the mixture. Advantageously, the mixture (e.g., “wash 3” as further described herein) may comprise a local, micro-environment dependent balance between cysteine and cystine that facilitates selective reduction of the antibodies. As further described herein, supplementary cystine may also be added to the mixture comprising the reducing agent.

The mixture comprising the reducing agent may be applied to the bound antibodies in a single step or in multiple steps. The solution comprising the reducing agent may be applied at a constant concentration or stepwise gradient.

Any suitable amount of reducing agent may be used. The amount may depend on the reaction conditions (e.g., type of reducing agent, type and/or amount of antibody, type of sample, % un-cysteinylated antibodies in the sample, type of chromatography material, length of reaction time, temperature and/or pH). A suitable amount of reducing agent will allow adequate un-cysteinylation of the antibodies, whilst limiting any fragmentation (e.g., overreduction) through disruption of conserved disulfide bonds elsewhere in the antibody.

In preferred embodiments, the molar ratio of reducing agent (e.g., cysteine) to antibodies (e.g., secukinumab, biosimilar or variant thereof) is between about 5:1 to about 100:1, typically between about 10:1 to about 40:1. Preferably, the molar ratio of the reducing agent (e.g. cysteine) to antibodies (e.g. secukinumab, biosimilar or variant thereof) is between about 20:1 to about 30:1.

In preferred embodiments, the molar ratio of reducing agent (e.g., cysteine) to antibodies (e.g., secukinumab, biosimilar or variant thereof) is optimized depending on % cysteinylation of unpaired cysteine in the antibodies of the initial sample. For example, a molar ratio of reducing agent (e.g. cysteine) to antibody (e.g., secukinumab, biosimilar or variant thereof) of about 20:1 to about 30:1 may be particularly advantageous when between about 80% to about 90% of the unpaired cysteines of the antibodies in the initial sample (e.g. harvest clarified cell fluid) are un-cysteinylated.

The mixture comprising the reducing agent may be contacted with the antibody for any suitable amount of time. The total duration of contact may depend on the reaction conditions (e.g., type and/or amount of reducing agent, type and/or amount of antibody, type of sample, % un-cysteinylated antibodies in the sample, type of chromatography material, temperature and/or pH). A suitable contact time will allow un-cysteinylation of the one or more unpaired cysteines, whilst limiting any fragmentation (e.g., overreduction) through disruption of conserved disulfide bonds elsewhere in the antibody.

In preferred embodiments, the mixture comprising the reducing agent (e.g., cysteine) may be contacted with the antibody (e.g., secukinumab, biosimilar or variant thereof) for between about 2 to 8 hours, e.g., about 5 or 6 hours in total.

In certain embodiments, the contact time of the reducing agent with the antibodies may be controlled by selecting an appropriate column flow rate as described herein. For example, higher flow rates and/or shorter contact times may be used with higher concentrations of the reducing agent.

As described further herein, the flow of the mixture comprising the reducing agent may also be paused one or more times to allow static contact of the mixture with the antibodies whilst bound to the chromatography material.

The mixture comprising the reducing agent may be contacted with the antibody at any suitable temperature. Typically, the temperature does not require any additional heating step. For example, the mixture comprising the reducing agent may be contacted with the antibody at room temperature (i.e., between about 15° C. to about 25° C.).

In preferred embodiments as further described herein, the sample is clarified cell culture fluid (typically comprising about 80% to 90% un-cysteinylated antibodies) and the chromatography material is protein A.

Redox Solutions

In certain embodiments, the mixture comprising the one or more reducing agents (e.g., cysteine) further comprises one or more oxidation reagents. As discussed above, the mixture may comprise one or more oxidation reagents as a result of spontaneous oxidization of the reducing agent in the mixture. In addition or alternatively, one or more oxidation reagents may be added separately to the reduction mixture.

For example, if a high molar ratio of reducing agent (e.g., cysteine):antibody (e.g., secukinumab, biosimilar or variant thereof) is used (i.e., an excess of reducing agent to antibody), then one or more oxidation reagents may be used to help mitigate the reductive power of the reducing agent.

In certain embodiments, the mixture comprises a set of reduction/oxidation (redox) reagents. For example, the mixture may comprise a thiol/disulfide redox pair. For example, the mixture may comprise reduced and oxidized glutathione, γ-glutamyl-cysteine, cysteinyl glycine, cysteine, cystine, N-acetylcysteine, cysteamine and/or dihydrolipoamide/lipoamide.

In preferred embodiments, the oxidation reagent comprises cystine. For example, cystine may be used as an oxidized redox partner to the sulfhydryl-group reducing agent (e.g., cysteine). In addition, cystine may inhibit thiol reducing enzymes (e.g., thioredoxin and thioredoxin reductase). Thus, addition of cystine may help keep conserved inter- and intra-molecular disulfide bonds in the antibody intact (thereby preventing fragmentation of the antibody).

Any suitable amount of oxidizing agent may be used in the mixture. The amount of oxidizing agent may vary depending on the reaction conditions (e.g., type and/or amount of reducing agent, type and/or amount of antibody, type of sample, % un-cysteinylated antibodies in the sample, type of chromatography material, temperature, length of reaction time, pH etc.)

In certain embodiments, the molar ratio of the sulfhydryl-group containing reducing agent (e.g. cysteine):oxidation agent (e.g. cystine) is between about 50:1 and 1:1, preferably between about 10:1 and 20:1.

In certain embodiments, one or more oxidizing reagents are not added to the mixture. In certain embodiments, cystine is not added to the mixture.

In certain embodiments, the mixture may or may not contain additional components. For example, the mixture may or may not contain a metal chelator (e.g., EDTA, DMSA, DMPS or the like).

In certain embodiments, the mixture further comprises EDTA and/or cupric sulfate (CuSO₄). For example, copper ions may increase the fragmentation rate of IgG molecules in the hinge region, likely due to reduction of disulfide bridges. The reaction is accelerated by increased concentrations of cupric ion and inhibited by EDTA. Conversely, EDTA and CuSO₄ may also act to inhibit thiol reducing enzymes (for example, thioredoxin and thioredoxin reductase).

Any suitable amount of EDTA may be used in the mixture. Depending on the reaction conditions discussed above, the molar ratio of reducing agent:EDTA may also vary. In some embodiments, the molar ratio of the reducing agent (e.g. cysteine):EDTA is between about 1:0.1 to about 1:10, preferably about 1:5.

In certain embodiments, the mixture does not contain EDTA and/or cupric sulfate (CuSO₄).

In certain embodiments, the mixture is isotonic.

In certain embodiments, the mixture includes a phosphate buffer, Tris buffer, acetate buffer, citrate buffer or the like. In certain embodiments, the mixture is at a pH of about 7.5 to 8.5, preferably about 7.3 to 8.2. For example, the pH of the mixture may be about 8.0.

In certain embodiments, the mixture comprising the reducing agent(s) may include an agent that reduces electrostatic interactions including salts, e.g., sodium salts, potassium salts, ammonium salts, citrate salts, calcium salts, magnesium salts and the like. Typically, however, the mixture comprising the reducing agent(s) lacks one or more salts (e.g., lacks NaCl).

In certain embodiments, the mixture comprising the reducing agent(s) comprises the same buffers used in any previous equilibration and/or wash buffer. However, the concentration and/or pH of the buffers may be different in the reduction mixture.

In preferred embodiments, the mixture (e.g., “wash 3” as described herein) is Tris-buffered. Typically, the mixture has a pH of about 7.3 to 8.2. By way of example, the mixture may comprise about 6 mM Tris, about 8.1 mM reducing agent (e.g., cysteine) with pH of about 8.0. Such a reduction mixture may be particularly advantageous, for example, where between about 80% to 90% of the antibodies in the initial sample (e.g. clarified cell culture fluid) are un-cysteinylated, the ratio of reducing agent (e.g. cysteine) to antibody (e.g. secukinumab, variant or biosimilar thereof) is between about 20:1 to about 30:1 and/or the reducing agent is contacted with the antibodies bound to chromatography material (e.g. protein A) for about 5 to 6 hours.

In the method of the invention, passing the mixture through the chromatography material allows contact of the redox reagents (e.g., reducing agent(s) and/or oxidation reagent(s)) with the antibodies whilst bound to the chromatography material.

The mixture may be passed through the chromatography material at any suitable flow rate. The flow rate may depend on the column size. The flow rate may be selected depending on the total contact time chosen for the mixture and antibodies and/or the molar concentration of reducing agent(s):antibody.

In certain embodiments, the mixture may be passed through the chromatography material (e.g., Protein A) at a flow rate between about 10 cm/h to about 1000 cm/h, preferably between about 300 cm/h to 400 cm/h (e.g. for large scale applications) or between about 25 cm/h to about 300 cm/h (e.g. for small scale applications). For example, the mixture may be passed through the chromatography material at a flow rate of about 50, 100 or 150 or 200 cm/h. Typically, the flow rate is about 100, 200, 300, 400 or 500 cm/h.

The total duration of contact of the antibodies with the mixture may vary depending on the reaction conditions being used (e.g., type and/or amount of reducing agent, presence of oxidizing agent, type and/or amount of antibody, type of sample, % un-cysteinylated antibodies in the sample, type of chromatography material, temperature, length of reaction time, pH, etc.)

In certain embodiments, the flow of the mixture through the chromatography material loaded with antibodies is paused one or more times. This allows static contact of the reducing agent and/or oxidation reagent with the antibodies whilst bound to the chromatographic material.

In certain embodiments, the flow of the mixture is paused two, three, four, five or more times. Typically, the flow of the mixture is paused up to three times. Each pause may be approximately the same or a different duration. Typically, each pause is about 40 minutes, about 60 minutes, about 80 minutes, about 100 minutes or about 120 minutes. This allows static contact of the reducing agent with the antibodies for a total of between about 2 to about 6 hours.

Preferably, the mixture comprising the reducing agent(s) is statically contacted with the antibodies whilst bound to the chromatography material for between about 5 to 6 hours in total.

Post-Reduction Washes

After the bound antibodies have been contacted with the mixture comprising one or more reducing agents, one or more post-wash buffers may be passed through the chromatography material. Advantageously, washing the bound antibodies removes any reducing agent or other unbound material. Typically, a single wash is sufficient to remove any unbound material prior to elution of the antibodies. Typically, at least 5×, 10×, 15×, 20× or more column volumes of the post-wash buffer are washed through the chromatography material.

Any suitable post-wash buffer may be used. Suitable buffers include phosphate buffer, Tris buffer, acetate buffer, citrate buffer or the like. The one or more post-wash buffer(s) may be adjusted to any desired pH e.g., using acetic acid or the like. Typically, the wash buffer is about pH 5.0 to about 6.0. For example, the wash buffer may be about pH 5.5.

In certain embodiments, the post-wash buffer(s) (e.g., “wash 4” as further described herein) comprise the same buffers used in any previous equilibration buffer and/or reduction mixtures (optionally at a different concentration and/or pH). Typically, however, the post-wash buffers are different from the equilibration buffer but comprise the same buffer agent used in the elution buffer. The post-wash buffer(s) may or may not contain an agent that reduces electrostatic interactions including salts, e.g., sodium salts, potassium salts, ammonium salts, citrate salts, calcium salts, magnesium salts and the like. Typically, the wash buffer(s) do not contain any salts.

In preferred embodiments, the post-wash buffer is an acetate buffer. Typically, the mixture has a pH of about 5.0 to 6.0. By way of example, the post-wash buffer may comprise about 50 mM Na—HAc, with pH of about 5.5.

The post-wash buffer(s) may be passed through the chromatography material at any suitable flow rate. The flow rate may depend on the column size. For example, the post-wash buffer(s) may be passed through the chromatography material at a flow rate between about 10 cm/h to about 1000 cm/h, preferably between about 300 cm/h to 400 cm/h (e.g., for large scale applications) or between about 25 cm/h to about 300 cm/h (e.g., for small scale applications). For example, the post-wash buffer(s) may be passed through the chromatography material at a flow rate of about 50, 100 or 150 or 200 cm/h. Typically, the flow rate is about 100, 200, 300, 400 or 500 cm/h.

The post-wash buffer(s) may be contacted with the chromatography material at any suitable temperature. Typically, the temperature is between about 15° C. to 25° C. degrees.

Elution of Antibodies

In certain embodiments, the method involves eluting the antibodies from the chromatography material in order to obtain an eluate.

Typically, the antibodies are removed from the chromatography material using one or more elution buffer(s). Any suitable buffer that allows dissociation of the antibodies from the chromatography material may be used. Typically, a single elution step is sufficient to elute the antibodies. However, multiple elution steps may be performed if required.

Any suitable elution buffer may be used. Suitable elution buffers include phosphate buffer, Tris buffer, acetate buffer, citrate buffer or the like. The one or more elution buffer(s) may be adjusted to any desired pH e.g., using acetic acid or the like. Typically, the elution buffer is low pH. Typically, the elution buffer is between about pH 3.2 to 4.2. For example, the elution buffer may be about pH 3.7.

In certain embodiments, the elution buffer(s) comprise the same buffers used in any previous, equilibration buffer, post-loading buffer, reduction mixtures and/or post-wash buffer (optionally at a different concentration and/or pH). Typically, however, the elution buffer(s) are different than at least the equilibration buffer(s), post-loading buffer(s) and/or reduction mixture and/or are the same as used in the previous wash.

Typically, the elution buffer(s) does not contain any salts.

In preferred embodiments, the elution buffer is an acetate buffer. Typically, the elution buffer has a pH of about 3.2 to 4.2. By way of example, the elution buffer may comprise about 50 mM Na—HAc (acetic acid), with pH of about 3.7.

The elution buffer(s) may be passed through the chromatography material at any suitable flow rate. The flow rate may depend on the column size. For example, the elution buffer(s) may be passed through the chromatography material at a flow rate between about 10 cm/h to about 1000 cm/h, preferably between about 300 cm/h to 400 cm/h (e.g., for large scale applications) or between about 25 cm/h to about 300 cm/h (e.g., for small scale applications). For example, the elution buffer(s) may be passed through the chromatography material at a flow rate of about 50, 100 or 150 or 200 cm/h. Typically, the flow rate is about 100, 200, 300, 400 or 500 cm/h.

The elution buffer(s) may be contacted with the chromatography material at any suitable temperature. Typically, the temperature is between about 15° C. to 25° C. degrees.

In certain embodiments, the eluted antibodies are also subject to a low pH viral inactivation step. Any suitable technique of viral inactivation may be applied, as well described in the art for commercial production of antibodies. For example, the eluted antibodies may be incubated at a low pH (e.g., between about 3.2 to 3.6, typically pH 3.4) for any suitable amount of time (e.g., between about 30 minutes to 2 hours, typically 60 minutes) before any subsequent neutralisation step.

Following elution (and optional low pH viral inactivation), the antibodies are typically neutralized. Any suitable technique of neutralizing the antibodies may be applied, as described in the art. For example, the eluted antibodies may be neutralized to a pH of about 7.4 to about 7.8. Typically, the eluted antibodies are neutralized using any suitable base (e.g., 1 M Tris).

Holding Step

In certain embodiments after the initial chromatography step (e.g., Protein A), a holding step is performed. Advantageously, the holding step may decrease any low molecular weight fragments (LMW) in the sample of eluted and/or neutralized antibodies.

Any suitable holding step may be performed. Typically, the sample is incubated prior to any further downstream steps of purifying the antibodies (e.g., additional chromatography steps such as AEX, Polisher technology (e.g., 3 M Polisher ST), CEX and/or MMC as further described herein). Without being bound to theory, it is understood the holding steps described herein advantageously allow LC fragments to reform with HHL fragments in the sample of antibodies.

As used herein, a “decrease” of any LMW may refer to a decreased (or eliminated) amount of any LMW in the sample of antibodies as compared to a sample that is not subject to any holding step as described herein.

Typically, the eluted (and/or neutralized) antibodies are incubated under conditions that decrease the amount of any LMW fragments in the sample of the eluted (and/or neutralized) antibodies. For example, the conditions further described herein may reduce the total LMW % in the sample of antibodies to about 10%, about 5%, about 4%, about 3%, about 2%, about 1.6%, 1.5%, 1.4%, 1.3%, 1.2%, 1.1%, 1%, 0.9%, 0.8%, 0.7%, 0.6%, 0.5%, 0.4%, 0.3%, 0.2%, 0.1% or less. Any suitable technique for determining total LMW % in a sample of antibodies may be used, including, for example, non-reduced capillary gel electrophoresis (CGE) or the like. The antibodies may be incubated under any suitable temperature that decrease the amount of any LMW fragments in the sample. For example, the antibodies may be incubated at a temperature of about 2° C. to about 25° C. (e.g., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C.). In certain embodiments, the antibodies are incubated at a low temperature (e.g., between about 2° C. to about 8° C., typically about 5° C.). Alternatively, the antibodies may be incubated at room temperature (e.g., between about 15° C. to about 25° C.). Advantageously, incubating at room temperature may act to reduce the time required for the incubation step.

The antibodies may also be incubated for any time sufficient to decrease the amount of any LMW fragments in the sample. The duration of the incubation step may depend, for example, on the temperature of the incubation step and/or whether the sample of antibodies is agitated (e.g., shaken or stirred) during incubation. Typically, the antibodies are incubated for between about 1 hour to about 4 days, e.g., about 6 to about 18 hours, or 24 hours to about 72 hours (e.g., 30 hours, 36 hours, 42 hours, 48 hours, 54 hours, 60 hours, etc).

In one embodiment, the antibodies are incubated for about 24 hours or more at room temperature. Alternatively, the antibodies may be incubated at a lower temperature (e.g., about 5° C.). In such embodiments, the antibodies may be agitated (e.g., stirred or shaken) to help reduce the amount of time required to decrease the amount of any LMW fragments in the sample.

In certain embodiments, the antibodies are first incubated at the temperature of interest then frozen at <-70° C. at the desired time.

The antibodies may be incubated in any suitable buffer. Typically, the buffer is the same as the buffer used to elute the antibodies from the chromatography material (e.g., protein A). For example, the buffer may be an acetate buffer neutralized to a pH of about 7.4 to 7.8 (e.g., a pH of about 7.5, 7.6, 7.7, or 7.8. Typically, the buffer comprises about 50 mM Na—HAc (acetic acid) at a pH of about 7.5.

Further Purification of Antibodies

In certain embodiments, the method comprises passing the harvested and/or eluted antibodies through one or more further chromatography materials.

In certain embodiments, protein A is used as the chromatography material for “on-column” reduction. In alternative embodiments, selective reduction is not required during the protein A chromatography step. In such embodiments, standard techniques of Protein A or the like can be performed as well described in the art, where incubation of the antibodies with a reducing agent is not required.

In any such embodiments, the selectively reduced antibodies can be subsequently passed through one or more anion exchange (AEX), cation-exchange (CEX) and/or multi-modal chromatography (MMC) materials as described herein. In certain embodiments, Polisher technology (e.g., 3 M Polisher ST or the like) is used to replace downstream AEX polishing columns). Typically, the antibodies are contacted with one more further chromatography materials under conditions that allow further isolation and/or purification of the antibodies from the initial sample.

As described herein, AEX, CEX and/or MMC purification typically includes the following steps performed sequentially: (1) equilibration of the relevant chromatography material using one or more equilibration buffer(s); (2) loading the antibody sample to be purified onto the chromatography material; (3) one or more wash step(s) using one or more wash buffer(s); and (4) elution of the antibody of interest using one or more elution buffer(s).

As used herein, an “equilibration buffer” is a buffer that is used to equilibrate the chromatography material (e.g., AEX, CEX and/or MMC) prior to loading the sample of antibodies onto the chromatography material.

As used herein, a “wash buffer” is a buffer that is passed over the chromatography material (e.g., AEX, CEX and/or MMC) following loading of the sample of antibodies onto the chromatography material.

As used herein, an “elution buffer” is a buffer that is used to elute the antibody of interest from the chromatography material (e.g., AEX, CEX and/or MMC).

In certain embodiments, the downstream processing steps may reduce or eliminate any low molecular weight protein fragments from the antibody sample. In certain embodiment, the downstream processing steps may reduce or eliminate any mono-cysteinylated antibodies. For example, re-oxidation of conserved cysteine-cysteine disulfide bonds may occur during downstream processing steps such as anion and/or cation exchange chromatography.

In certain embodiments, AEX chromatography is used to remove and/or reverse any fragmentation of the antibody resulting from the Protein A step and/or resulting from low pH viral inactivation that may be performed following Protein A. Commercially available anion-exchange materials, include, for example, Sartobind STIC, Porous HQ, Eshmuno Q or the like. In alternative embodiments, Polisher technology (e.g., 3 M Polisher ST) may replace such downstream AEX steps.

In certain embodiments, CEX and/or MMC is used to remove and/or reverse any acidic variants and/or glutathionylated species in the sample of antibodies.

As used herein, a “cation exchange material” refers to a solid phase that is negatively charged and has free cations for exchange with cations in an aqueous solution passed over or through the solid phase. The charge may be provided by attaching one or more charged ligands to the solid phase, e.g., by covalent linking. Alternatively or additionally, the charge may be an inherent property of the solid phase (e.g., silica has an overall negative charge). Commercially available cation exchange materials include carboxy-methyl-cellulose, BAKERBOND ABX™, sulphopropyl (SP) immobilized on agarose (e.g., SP-SEPHAROSE FAST FLOW™, SP-SEPHAROSE FAST FLOW XL™ or SP-SEPHAROSE HIGH PERFORMANCE™, from GE Healthcare), CAPTO S™ (GE Healthcare), FACTOGEL-SO3™, FACTOGEL-SE HICAP™, and FRACTOPREP™ (EMD Merck), sulphonyl immobilized on agarose (e.g., S-SEPHAROSE FAST FLOW™ from GE Healthcare), and SUPER SP™ (Tosoh Biosciences), POROS 50 HS® chromatography resin) or the like.

As used herein, an “acidic variant” (or acid charge variant) refers to any acidic species in the sample of antibodies. For example, during chromatographic analysis, acidic variants may elute earlier than the main peak during CEX (or later than the main peak during AEX). Causes for the formation of acidic variants in monoclonal antibodies may include sialic acid, deamidation, non-classical disulfide linkage, trisulfide bonds, high mannose, thiosulfide modification, glycation, modification by maleuric acid, cysteinylation, glutathionylation, reduced disulfide bonds, non-reduced species or fragments or the like. See, for example, Du et al, Mabs, 2012, 4(5) 578-585, herein incorporated by reference.

As used herein, “Glutathionylation” refers to the reversible addition of a proximal donor of glutathione to thiolate anions of cysteine residues in an antibody. For example, glutathionylated species may be linked to the non-paired cysteine in secukinumab's light chain CDR3.

In certain embodiments, the invention provides a method of removing or eliminating acidic variants and/or glutathionylation in a sample of antibodies, wherein the method comprises:
(a) passing the sample of antibodies through one or more chromatography material(s) (e.g., CEX, AEX and/or MMC), thereby binding the antibodies to the chromatography material(s);
(b) contacting the bound antibodies with a wash buffer; and
(c) eluting the antibodies from the chromatography material.

As discussed herein, these methods can be performed regardless of whether selective reduction of the one or more unpaired cysteines of the antibody has been performed (a) during upstream processing steps of cell culture and/or (b) during “on-column” reduction in any preceding chromatography steps (e.g., Protein A).

Typically, the pH of the wash buffer is optimised depending on the amount of antibody being loaded onto the chromatography material (e.g., CEX, AEX and/or MMC). Typically, the pH of the wash buffer is higher than the pH of any equilibrium buffer being used (e.g., at least about pH 7.0 or more).

In certain embodiments, step (a) comprises binding antibodies to the chromatography material at a loading capacity of about 10 g/L or less (e.g., 9.9 g/L, 9 g/L, 8 g/L, 7 g/L, 6 g/L, 5 g/L, 4 g/L, 3 g/L, 2 g/L, 1 g/L, 0.9 g/L, 0.8 g/L, 0.5 g/L or less).

In alternative embodiments, step (a) comprises binding antibodies to the chromatography material at a loading capacity of more than about 10 g/L, 20 g/L, 30 g/L, 40 g/L, 50 g/L, 60 g/L, 70 g/L, 80 g/L, 90 g/L, 100 g/L or more. In certain embodiments, the loading density of antibodies onto the chromatography material (e.g., CEX, AEX and/or MMC) is about 10 g/L resin (or less).

In such embodiments, the method of the invention may comprise:
(a) passing the antibodies through one or more chromatography material(s) (CEX, AEX and/or MMC), thereby binding the antibodies to the chromatography material(s);
(b) contacting the bound antibodies with a wash buffer, wherein the wash buffer is at a pH above the isoelectric point (pI) of the antibodies in the sample (e.g., at a pH of between about 8.8 to 9.0 (e.g., a pH of about 8.9); and
(c) eluting the antibodies from the chromatography material.

In alternative embodiments, the loading density of antibodies onto the chromatography material (CEX, AEX and/or MMC) is more than about 10 g/L resin. In such embodiments, the method of the invention may further comprise:
(a) passing the antibodies through one or more chromatography material(s) (CEX, AEX and/or MMC), thereby binding the antibodies to the chromatography material(s);
(b) contacting the bound antibodies with a wash buffer, wherein the wash buffer is at a pH which is the same or below the isoelectric point (pI) of the antibodies in the sample (e.g., about 7.0 to 8.4, preferably about 8.0; and
(c) eluting the antibodies from the chromatography material.

In certain embodiments, the loading density of antibodies onto the chromatography material is about 10 g/L resin. In these embodiments, the method of the invention may further comprise:
(a) passing the antibodies through one or more CEX chromatography material(s), thereby binding the antibodies to the chromatography material(s);
(b) contacting the bound antibodies with a wash buffer, wherein the wash buffer is at a pH which is above the isoelectric point (pI) of the antibodies in the sample (e.g., at a pH of about 8.9 or more); and
(c) eluting the antibodies from the chromatography material.

In certain embodiments, the loading density of antibodies onto the chromatography material is between about 20-30 g/L resin. In these embodiments, the method of the invention may further comprise:
(a) passing the antibodies through one or more CEX chromatography material(s), thereby binding the antibodies to the chromatography material(s);
(b) contacting the bound antibodies with a wash buffer, wherein the wash buffer is at a pH which is the same or below the isoelectric point (pI) of the antibodies in the sample (e.g., at a pH about 7.0 to 8.4, preferably between about 8.0-8.2); and
(c) eluting the antibodies from the chromatography material.

In certain embodiments, the loading density of antibodies onto the chromatography material is between about 20-30 g/L resin. In these embodiments, the method of the invention may further comprise:
(a) passing the antibodies through one or more MMC chromatography material(s), thereby binding the antibodies to the chromatography material(s);
(b) contacting the bound antibodies with a wash buffer, wherein the wash buffer is at a pH which is the same or below the isoelectric point (pI) of the antibodies in the sample (e.g., at a pH about 7.0 to 8.0, preferably about 7.5); and
(c) eluting the antibodies from the chromatography material.

Typically, the antibodies are anti-IL-17 antibodies as described herein. For example, the antibodies are preferably anti-IL-17 (i.e., IL-17A) antibodies (e.g., secukinumab). As used herein, “anti-IL-17 antibodies” include any antibodies (or antigen-binding fragments thereof) capable of specifically binding to IL-17. Typically, the sample of antibodies is obtained following any earlier “on-column” reduction steps (e.g., during protein A chromatography) as described herein.

Typically, the additional chromatography material used for further purification of antibodies is CEX. Typically, the pH wash as described herein is optimized for a downstream purification step using CEX.

In the methods of purifying antibodies described herein, the chromatography material (e.g., CEX, AEX and/or MMC) is typically contacted with one or more equilibration buffers prior to loading the sample comprising the antibodies onto the material.

Any suitable equilibration buffer may be used. Suitable equilibration buffers include phosphate buffer, Tris buffer, acetate buffer, citrate buffer or the like. The buffer may be adjusted to any desired pH e.g., using acetic acid or the like. Typically, the equilibration buffer is about pH 5.0 to about pH 6.0. For example, the equilibration buffer may be about pH 5.5. In preferred embodiments, the equilibration buffer is an acetate buffer. By way of example, the equilibration buffer may comprise about 50 mM Na-Acetate-HAc, with pH of about 5.5.

Following equilibration, the sample of antibodies is loaded onto the chromatography material. The sample for loading is typically adjusted to the same ionic strength and/or pH as the equilibration buffer prior to loading of the sample onto the chromatography material. For example, the loading buffer is also typically about pH 5.0 to about pH 6.0 (e.g., about pH 5.5) and/or an acetate buffer. Any suitable loading capacity of antibodies may be used, which may depend, for example, on the type of chromatography material being used.

In certain embodiments, the sample of antibodies is incubated with the chromatography material (e.g., CEX, AEX or MMC) under conditions that allow binding of the antibodies to the chromatography material. For example, the antibodies may be incubated with CEX, AEX and/or MMC resin using any standard conditions described in the art.

After the antibodies have been loaded onto the chromatography material (e.g., CEX, AEX or MMC), one or more wash buffers is passed through the chromatography material. Typically, one, two, three or more wash buffers are passed over the chromatography material.

Advantageously, washing the bound antibodies with one or more wash buffers as described herein removes unbound material from the chromatography material including acidic variants and/or glutathionylated antibodies in the sample. As described herein, the pH of the wash buffer may be optimized depending on the amount of antibodies being loaded onto the chromatography material.

In preferred embodiments, about 10 g/L of antibodies are loaded onto the chromatography material (e.g., CEX). In such embodiments, the wash buffer (e.g., Tris buffer) is at a pH of about 8.8 to 9.0 (e.g. about 8.9). The pH of the wash buffer may depend on the loading capacity of the chromatographic column. By way of example, the wash buffer may comprise about 50 mM Tris, with pH of about 8.9. In such embodiments, washing at a high pH during chromatography (e.g., CEX) enables the acidic variants of the antibody and/or the glutathionylated species to be significantly and efficiently decreased, as well as reducing the level of cysteinylated species and low molecular weight species in the sample.

Any suitable technique for determining the pI of acidic variants and/or glutathionylated antibodies in the sample of antibodies may be used. Suitable techniques include, for example, isoelectric focusing (IEF) gel electrophoresis, capillary isoelectric focusing (cIEF) gel electrophoresis or the like. Acidic species have lower apparent pI as compared to main species of antibodies when analysed using IEF methods. Typically, at least 3×5×, 10×, 15×, 20× or more column volumes of the wash buffer are washed through the chromatography material. For example, at least about 3× column volumes of the buffer may be washed through the chromatography material.

In certain embodiments, a loading capacity of 10 g/L resin in used. In such embodiments, at least about three column volumes of pH 7.4 (pre-) wash buffer, at least about three column volumes of pH 8.9 wash buffer, and at least three column volumes of pH 7.4 (post-) wash buffer may be passed over the chromatography material loaded with the antibodies of interest.

Any suitable wash buffer(s) may be used. Suitable wash buffers include phosphate buffer, Tris buffer, acetate buffer, citrate buffer or the like. Typically, the wash buffer is a Tris buffer.

Typically, the wash buffer is at pH of about 7.4 to about 10 (e.g., a pH of about 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, or 9.9). The pH of the wash buffer may be adjusted depending on the loading capacity of the chromatographic material. For example, where about 10 g/L of antibodies (or less) are loaded onto the chromatography material, the pH of the wash buffer may be above the isoelectric point of the antibodies (e.g., between about pH 8.8 to 9.0, preferably about 8.9). Alternatively, if more than about 10 g/L of antibodies are loaded onto the chromatography material (preferably between about 20 g/L to about 30 g/L), the pH of the wash buffer may be the same or below the isoelectric point of the antibodies (e.g., between about pH 7.0 to 8.4, preferably about 8.0).

In certain embodiments where more than one wash buffer is used, the bound antibodies may be contacted with one or more additional wash buffers before and/or after the step of contacting the bound antibodies with an optimised (e.g., high pH) wash buffer.

Any suitable pre-wash or post-wash buffers may be used. In embodiments where a pre-wash is performed prior to the optimized wash step and a post-wash is performed after the optimized wash step, the pre-wash and post-wash buffers that are used are typically the same.

Suitable pre- and/or post-wash buffers include phosphate buffer, Tris buffer, acetate buffer, citrate buffer or the like. The pre- and/or post wash buffer(s) may be adjusted to any desired pH. Typically, the pre- and/or post wash buffer is about pH 7.0 to about 8.0. For example, the pre- and/or post wash buffer may be about pH 7.4. In preferred embodiments, the pre- and/or post-wash buffer is a Tris buffer. By way of example, the pre- and/or post-wash buffer may comprise about 50 mM Tris, with pH of about 7.4. In certain embodiments, a re-equilibration step is performed after the chromatography wash steps and prior to elution of the antibodies from the chromatography material. As used herein, a re-equilibrium (or regeneration) buffer may regenerate the chromatography material (e.g., AEX, CEX and/or MMC) such that it can be re-used. The re-equilibrium buffer has a conductivity and/or pH as required to remove substantially all contaminants and the antibody of interest from the chromatography material.

Any suitable re-equilibration buffer may be used. Typically, the re-equilibration buffer is the same as the equilibration buffer. For example, the re-equilibration buffer may be about pH 5.5. In preferred embodiments, the re-equilibration buffer is an acetate buffer. By way of example, the equilibration buffer may comprise about 50 mM Na-Acetate-HAc, with pH of about 5.5.

In certain embodiments, the method involves eluting the antibodies from the chromatography material in order to obtain an eluate. Typically, the antibodies are removed from the chromatography material using one or more elution buffer(s).

Any suitable buffer that allows dissociation of the antibodies from the chromatography material may be used. For example, elution of the antibody may be achieved by increasing the conductivity or ionic strength. Typically, the conductivity of the elution buffer is greater than about 10 mS/com. Increased conductivity may be achieved by including a relatively high salt concentration in the elution buffer. Exemplary salts include, for example, sodium acetate, sodium chloride, potassium chloride or the like.

Typically, a single elution step is sufficient to elute the antibodies. However, multiple elution steps may be performed if required. In certain embodiments, the chromatography material column may be washed with the first wash buffer and/or further equilibrated with the equilibration buffer before contacting the chromatography material with the elution buffer.

Any suitable elution buffer may be used. Suitable elution buffers include phosphate buffer, Tris buffer, acetate buffer, citrate buffer or the like. The one or more elution buffer(s) may be adjusted to any desired pH e.g., using acetic acid or the like. Typically, the elution buffer is low pH. Typically, the elution buffer is between about pH 5.0 to 6.0. For example, the elution buffer may be about pH 5.5. In preferred embodiments, the elution buffer is an acetate buffer. The elution buffer(s) may or may not contain an agent that reduces electrostatic interactions including salts, e.g., sodium salts, potassium salts, ammonium salts, citrate salts, calcium salts, magnesium salts and the like. Typically, the elution buffer(s) contains salts. By way of example, the elution buffer may comprise about 50 mM Na-Acetate-HAc, 500 mM NaCl, with pH of about 5.5. Typically, further purification of the antibodies as described herein reduces the % amount of any acidic variants in the sample to less than about 30%, 25%, 20%, 15%, 12.5% or less. Any suitable technique for determining the % acidic variants in a sample of antibodies may be used. Techniques for evaluating % acidic variants in an antibody sample include, for example, carboxypeptidase B (CpB) analysis or the like.

Typically, further purification of the antibodies as described herein reduces the glutathionylation relative area (%) to about 3%, about 2%, about 1%, about 0.5% or less. Any suitable technique for determining % glutathionylation in a sample of antibodies may be used. Techniques for evaluating glutathionylation relative area (%) in an antibody sample include, for example, intact mass spectrometry or the like.

In certain embodiments, the one or more downstream processing steps may comprise a filtration step (e.g., viral filtration step).

In certain embodiments, the methods of the invention further comprise formulating a pharmaceutical composition comprising the purified antibodies. For example, the pharmaceutical composition may be a liquid or a lyophilized composition.

Purified Antibodies

The invention also provides a purified preparation of antibodies obtainable by any method as described herein.

Advantageously, the unpaired cysteines of the antibodies are un-cysteinylated (i.e., free) in at least 90%, at least 95%, at least 96%, at least 97%, at least 98% or at least 99% or more of the resulting antibodies. The level of cysteinylation of unpaired cysteines (e.g., in the one or more CDRs) may be measured using any suitable technique including mass spectrometry and the like. For example, HIC-HPLC may be used to determine % de-cysteinylation of antibodies as described herein.

In certain embodiments, the antibodies of the invention have improved levels of un-cysteinylation as compared to reference approved antibodies. For example, HIC-HPLC or the like may be used to distinguish an antibody of the invention from any reference antibody having greater levels of cysteinylation. Typically, the antibodies of the invention have at least 5%, 10%, 15%, 20%, 25% less cysteinylation of unpaired cysteines as compared to a reference antibody.

Advantageously, the % level of intact antibodies may be at least about 90%, 91%, 92%, 93%, 94%, 95%, 96%, 97%, 98%, 98.5%, 99% or more. The level of intact antibodies may be measured using any suitable technique. For example, sodium dodecyl sulfate capillary electrophoresis (CE-SDS) or Capillary Gel Electrophoresis (CGE) may be used to determine the % level of intact antibodies using standard techniques in the art. Typically, the antibodies of the invention have at least 2%, 5%, 10%, 15%, 20%, 25% or less low molecular weight (LMW) fragments as compared to a reference antibody.

In certain embodiments, CE-SDS, CGE or the like may be used to distinguish an antibody of the invention from any prior art antibody having lower % level of intact antibodies.

In certain embodiments, the antibodies eluted after the on-column reduction step (e.g., Protein A) are at least about 95% intact. The antibodies resulting from further downstream processing steps (e.g., incubation of Protein A eluate, CEX, AEX and/or MMC as described herein) may be at least about 96%, 97%, 98%, 99%, 99.5%, 99.99% or more intact.

In certain embodiments, the invention provides a purified preparation of secukinumab, biosimilar or variant thereof wherein CysL97 is un-cysteinylated in at least 95%, 96%, 97%, 98%, 99% or more of the antibodies following contact with the mixture comprising one or more reducing agent.

In certain embodiments, the invention provides a purified preparation of secukinumab, biosimilar or variant thereof wherein the % level of intact antibodies is at least about 95%, 96%, 97%, 98.0%, 98.5%, 99% or more.

Advantageously, the methods of the invention allow the purification and isolation of antibodies that retain biological activity and/or structure as compared to reference approved antibodies.

In certain embodiments, the % level of activity of the purified antibodies is at least about 90%, 91%, 92%, 93%, 94%, 95%, 96%, 97%, 98%, 99% or more. The % level of activity of the antibodies may be measured using any suitable technique including ELISA based assay, cell-based assay, cystamine-CEX and the like (see, e.g., WO2006/013107; WO2007/117749; Shen and Gaffen (2008) Cytokine. 41(2): 92-104) as incorporated herein by reference.

In certain embodiments, the antibodies retain at least about 90%, 91%, 92%, 93%, 94%, 95%, 96%, 97%, 98%, 99% or more binding ability to IL-17 as compared to a sample of reference antibodies. Typically, the antibodies have comparable or improved characteristics as compared, for example, to conventional formulations of secukinumab (e.g., Cosentyx).

Antibody Compositions and Uses

In certain embodiments, the antibodies obtained by the methods described herein are prepared for subsequent uses in diagnostic assays, immunoassays and/or pharmaceutical compositions. For example, the antibodies obtained by the methods described herein (e.g., secukinumab, biosimilar or variant thereof) may form an active ingredient of a pharmaceutical composition and/or medicament.

In certain embodiments, the antibodies of the invention have increased storage stability and/or decreased tendency to aggregate as compared to any untreated control antibodies. As described herein, an “untreated control” is an antibody produced by an equivalent method but lacking the step of selective reduction as described herein.

In certain embodiments, the antibodies maintain activity after storage for a period about 1, 2, 3, 6, 12, 18, 24, 48, 60 months or more. The antibodies may be stored in substantially hydrated or non-hydrated form. Typically, the antibodies are stored at 4° C., 0° C., −20° C., −70° C. or less.

The antibodies of the invention may be formulated into pharmaceutical compositions for administration to any subject (including humans). The compositions may comprise any pharmaceutically acceptable excipient or carriers. The compositions may be administered by any suitable method (e.g., subcutaneous, intravenous, or the like).

In certain embodiments, the methods may further comprise administering the antibodies to a subject. In certain embodiments, the antibodies are for use in therapy. For example, the purified preparation of antibodies (e.g., secukinumab, biosimilar or variant thereof) may be used in the treatment of autoimmune disorders such as active psoriatic arthritis, ankylosing spondylitis, active non-radiographic axial spondyloarthritis and the like.

### EXAMPLES

In the following, the invention will be explained in more detail by means of non-limiting examples of specific embodiments.

### Example 1—De-Cysteinylation of Antibodies During Cell Culture

To generate the data presented in FIGS. 1 and 2, 26 cultures in bioreactors 250 mL were conducted over two rounds. A design of experiment using the statistical tool MODDE was generated to study the impact of the seeding density, the presence and timing of temperature shift and pH shift and the culture duration on the cysteinylation levels (measured by HIC-HPLC). The design of experiment was generated in full factorial mode, to be able to estimate the impact of the parameters' potential interactions.

A condition selected as a control for comparison was performed (in addition to the design of experiment suggested by the software) with the following parameters:
seeding density at 0.2×10⁶ cells/mL;
Temperature shift from 37° C. to 33° C. at day 10 of culture;
pH shift from 7.1±0.3 to 6.8±0.3 at day 11 of culture (e.g., to maintain the cells at a pH of between about 6.8 to 7.1 over the course of the cell culture).

In the first round, the levels tested for each parameter is presented in Table 1 below:

TABLE 1
Upstream parameters tested for de-cysteinylation

| Factors | Levels tested |
|---|---|
| Target Seed Density | 0.2, 0.4 and 0.6 mio cells/mL |
| pH Shift from 7.1 to 6.8 ± 0.3 | None (0), day 9, 10 and 11 |
| Temp shift from 37 to 33° C. | None (0), day 9, 10, 11 and 12 |
| Culture Duration | Day 14 and 17 |

All cultures were kept until day 17, but a sampling followed by analysis was performed at day 14 to evaluate the impact of the culture duration.

Output (i.e. cysteinylation levels) were fed back in the MODDE design to generate the contour plot presented in FIG. 1.

The presence of temperature shift and pH shift can be identified in FIG. 1 as the parameters having the most of an impact: with both shifts performed (4 plot on top right), regardless of the days tested, the levels of de-cysteinylation obtained could easily reach >80%. The culture duration is also significantly impacting the cysteinylation levels, that decrease over time. The impact of the target seeding density is less significant compared to the others. This first round of experiment showed the importance of decreasing the temperature during the process, maintaining a low pH at the end of the process and indicated that a longer culture duration could decrease the cysteinylation levels.

Those observations were confirmed in the second round of experiment, where a different pH setpoint and deadband (6.95±0.15, without shift) were tested, combined with different seeding densities (0.2, 0.4 and 0.6 mio cells/mL) and always in the presence of a temperature shift (on day 10 or 12 of culture).

The data were compiled with those generated in round 1, and FIG. 2 display the results obtained when fixing the culture duration at 17 days (the higher duration tested in those experiments and the one with the lower cysteinylation levels in round 1) and keeping only the conditions with a pH shift and temperature shift from round 1.

As presented in FIG. 2, according to the model thus obtained the de-cysteinylated levels would be >90% just by fixing those parameters, regardless of the pH setpoint, dead band or the seeding density, which confirm the round 1 conclusions. This second plot allows a better view on the impact of the pH setpoint and dead band (lower cysteinylation with lower pH and tight dead band).

### Example 2—De-Cysteinylation Using On-Column Cysteine Washing

Antibodies secreted from Chinese Hamster Ovary (CHO) cell culture have a proportion of cysteinylated forms. In order to de-cysteinylate some or all of the antibodies, a study was conducted wherein the de-cysteinylation step was part of a standard chromatography step.

Protein A affinity chromatography of the CHO clarified cell culture was performed with addition of a third washing step (wash 3) containing various amount of cysteine either alone or in combination with cystine or EDTA at pH 8.0. The CHO clarified cell cultures were obtained by centrifugation and filtration of the CHO cell culture at day 14 produced in 250 mL bioreactors. The material was then called Protein A load and used as an input material. After freezing, Protein A loads were stored at <−70° C. and thawed less than 24 h prior to Protein A purification. Chromatography steps were performed at room temperature using an ÄKTA Pure FPLC system with Unicorn software (Cytiva).

The chromatography purification process is detailed in Table 2. Flow rates were adapted to the column size to reach a residence time of 4 minutes during the loading. The flow rates of the other steps were kept identical to the loading flow rate.

TABLE 2
Protein A chromatography conditions

| | Column | |
|---|---|---|
| | Volume | |
| Step | (CV) | Buffer |
| Equilibration | 10 | 50 mM Tris-Acetic acid, 150 mM NaCl, pH |
| | | 7.4 |
| Load | N/A | CHO clarified cell culture expressing the |
| | | antibody of interest |
| Wash 1 | 5 | 50 mM Tris-Acetic acid, 150 mM NaCl, pH |
| | | 7.4 |
| Wash 2 | 5 | 50 mM Tris-Acetic acid, 1M NaCl, pH 7.4 |
| Wash 3 | 3 CV + | Cysteine with or without cystine or EDTA |
| (except for | static and | solution at different concentration, pH 8.0 |
| Control | dynamic | |
| conditions) | incubation | |
| Wash 4 | 5 | 50 mM Sodium Acetate-Acetic acid, pH 5.5 |
| Elution | 8 | 50 mM Sodium Acetate-Acetic acid, pH 3.7 |

The eluate fraction was neutralized with 1 M Tris base to pH 7.4-7.7.

A proof of concept was carried out using the protocol in Table 2 and using the following conditions:
The ratio cysteine:antibody in mol/mol was 73:1, wash 3 solution was: 11.2 mM cysteine, 2 mM EDTA pH 8.0;
Wash 3 step was performed by renewing the washing solution 4 times with 1 Column Volume. In between these solution renewals a static incubation of 60 min was applied (4 pauses of 60 min).

Protein A column was a commercially available 5 mL prepacked column of MabSelect SuRe LX resin (Cytiva), prepacked column was loaded to a capacity of 63% of the DBC (10% breakthrough, 4 min residence time), thus 28 mg antibody/mL resin. The total duration of dynamic contact with cysteine was 15 min. The total duration of static contact with cysteine was 240 min.

Results

The level of cysteinylation was analysed by intact mass spectrometry. It resulted that the level of cysteinylation was decreased from control (without cysteine wash) compared to sample after the run (after cysteine wash). The mAb with two cysteinylated sites (2 Cys) was no longer detected; the mAb with a single cysteinylated site (1 Cys) was reduced from about 20% to about 5%.

FIG. 3 shows the comparison of two samples after a protein A chromatography step with or without the cysteine wash treatment. The samples were submitted to deglycosylation and decarboxylation to remove the N-glycans and C-terminal lysines before being analyzed by LC-MS. Main peak corresponds to the intact antibody free of cysteine, corresponding to a theoretical molecular mass of secukinumab lacking a C-terminal lysine from each heavy chain (i.e. 147688.68 Da). The peak labelled 1 Cys corresponds to the antibody containing one single cysteinylated C97 and one free cysteine. The peak 2 Cys corresponds to the antibody containing two cysteinylated C97.

As a result of this study, it was proven that an on-column treatment with a cysteine solution allowed de-cysteinylation of the antibodies light chains. As a drawback of this method, a high level of fragmentation was measured by capillary gel electrophoresis (CGE), 9.1% total low molecular weight (LMW), including 2.8% light chains (LC). Notably, this treatment did not affect the recovery of the protein A chromatography step compared to the runs without cysteine washing treatment.

Design of Experiments (DOE) was carried out to screen for parameters allowing de-cysteinylation of the molecule while maintaining other physico-chemical parameters constant, mainly the LMW level.

### Example 3: Effect of Different Factors on the On-Column Treatment

DOE1 was performed to evaluate the influence of three main factors on the de-cysteinylation of the antibodies: the ratio of mol cysteine in the wash solution to mol of antibody bound to the column; the total duration of the static incubation on-column (time); the ratio cysteine:EDTA in washing 3 solution.

The assays were performed in accordance to the descriptions of Example 2 and Table 2. Loadings were performed using approximately 47 mg antibody/mL resin (80% DBCqb10). Protein A chromatography steps were performed on 1 mL prepacked column of Praesto® Jetted A50 (Purolite®).

During additional post loading washing (Wash 3) containing cysteine at pH 8.0, 3 pauses of identical duration were performed for static incubation of the antibody mixture bound to the Protein A resin with Cysteine buffer, between which, 1 CV of washing 3 solution was passed through the column (Table 2). The eluate fractions were neutralized to pH 7.4-7.7 using 1 M Tris-base.

The evaluated input factor levels are shown in Table 3.

TABLE 3
DOE1 input factors

| | | Lower | Medium | Upper |
|---|---|---|---|---|
| Name | Unit | level | level | level |
| Ratio cysteine:antibody | mol:mol | 40 | 70 | 100 |
| (Cys:mAb) | | | | |
| Duration of the static | h | 2 | 4 | 6 |
| incubation (time) | | | | |
| Ratio Cysteine:EDTA | mM:mM | 0 | 5 | 10 |
| (cys:EDTA) | | | | |

The duration of static incubation was divided equally in three periods of static contact (pauses). For the lower, middle and upper levels, their durations were respectively: 40 minutes; 80 minutes and 120 minutes. The design of experiment was a full factorial design with 3 center points. Two control runs without cysteine wash were performed before and after the set of 11 runs. The experimental design plan and results is given in Table 4. DOE results were analyzed using jmp (SAS) software in combination with Minitab software (Minitab, Inc). Responses evaluated were: % Uncysteinylated species (% Uncys) measured by HIC-HPLC and the % low molecular weight (LMW) measured by CE-SDS.

Results

TABLE 4
Experimental plan of DOE1

| | FACTORS | | | | |
|---|---|---|---|---|---|
| | Ratio | | Ratio | | |
| | cys:mAb | Time | cys:EDTA | Uncysteinylated | LMW |
| Run Order | (mol:mol) | (h) | (mol:mol) | species (%) | (%) |
| CTRL initial | 0 | 0 | 0 | 21.39 | 3.01 |
| 1 | 40 | 2 | 10 | 47.52 | 17.13 |
| 2 | 100 | 2 | 10 | 74.67 | 82.99 |
| 3 | 70 | 4 | 5 | 81.28 | 63.42 |
| 4 | 70 | 4 | 5 | 83.35 | 56.52 |
| 5 | 100 | 6 | 0 | 91.52 | 39.45 |
| 6 | 40 | 6 | 10 | 85.22 | 21.89 |
| 7 | 100 | 6 | 10 | 89.31 | 67.56 |
| 8 | 40 | 6 | 0 | 84.00 | 11.95 |
| 9 | 100 | 2 | 0 | 75.14 | 73.66 |
| 10 | 70 | 4 | 5 | 76.57 | 47.90 |
| 11 | 40 | 2 | 0 | 54.19 | 26.62 |
| CTRL final | 0 | 0 | 0 | 24.28 | 3.21 |

In general, the data showed that the percentage of Uncysteinylated species was increased for all run conditions, compared to the control runs without cysteine washing (having a mean of 22.84% uncysteinylated species). However, some runs had a lower level than others. An example of Hydrophobic Interaction Chromatography-High Pressure Liquid Chromatography (HIC-HPLC) chromatograms overlay is displayed in FIG. 4.

FIG. 4 is an example of HIC-HPLC result comparing the profile without cysteine washing (in black) and after the cysteine washing (in blue). Samples were diluted in miliQ water to 0.4 mg/ml and eluted through a gradient 2 M Ammonium Sulfate, 100 mM Sodium Phosphate pH 7.0-100 mM Sodium Phosphate pH 7.0, 10% Acetonitrile. The profile in blue corresponds to run 1 (ratio Cys:mAb of 40 and time 6 h). It appears that peak 2 (1× cysteinylated) and peak 3 (2× cysteinylated) were decreased in profit of the main peak (de-cysteinylated antibody).

The data also showed that the LMW level after the step were above 10% for all the runs, compared to around an average of 3.11% for the control runs, without the cysteine wash. This treatment resulted in an important de-cysteinylation of the antibodies (from an average of 22.84% Uncysteinylated without treatment, up to more than between 47% to 91.5% depending on the conditions tested). However, this treatment resulted in over-reduction of the material.

The results showed that the runs with 2 hours of static contact (time) had the lower level of uncysteinylated species after the treatment and the runs with 6 hours had the higher level demonstrating that the static incubation increased the efficiency of the treatment. Among the runs with the same static duration (for example runs 1, 2, 9 and 11) the lower ratio Cys:mAb gave the lower level of uncysteinylated species (runs 1 and 11). For the percentage of LMW species, they were more important for the higher ratio Cys:mAb, and the time looked to have less impact.

FIG. 5 shows that the points are closed to the fitted line with narrow confidence bands, demonstrating a good correlation between the model and the data generated. The mathematical model showed good fit, for both responses, with a R² of 0.98 for the Uncysteinylated species percentage (by HIC-HPLC) and a R² of 0.88 for the LMW species percentage (by CGE).

Table 5 (Effects summary) for both uncysteinylated and LMW responses confirmed the impacts of the most critical factors identified above:time, ratio cys:mAb and the interaction Ratio Cys:Mab (mol:mol)*Time (h) for the % Uncys. Indeed, the LogWorth exceeded 2 and p-values were ≤0.01 for these three factors. The ratio Cysteine:EDTA was found not significant by the model. Mainly ratio Cys:mAb was identified as impacting the LMW % response. No main interactions between factors were identified for the LMW % response. The ratio of Cysteine:EDTA is not significant and does not explain the variability of both responses (p-values >0.1).

As the statistical model for the design of experiment showed good fit, it was possible to use contour plots (FIG. 6) to understand the main influence of the two most impactful factors:time and ratio of cysteine:antibody on both responses. The contour plots (FIG. 6) illustrate the effect of the ratio cys:mAb and the time on the decysteinylation and the creation of fragments (LMW). The model indicated that a percentage of uncysteinylated species equal or higher than 70% is reached for a static incubation duration (time) above 5 hours for ratios cysteine:mAb<50 mol/mol, whereas the LMW % stays lower than 40% for this ratio and time values.

To optimize de-cysteinylation and limit fragmentation, the model indicated that the factors could be set at the lowest level of ratio cysteine:mAb (40 mol:mol) and the highest level of contact duration of the wash (6 h), corresponding to the left upper part of the design space studied (FIG. 6).

Based on the results of DOE1, a new DOE, called DOE2 was performed to evaluate a new design space of input factors by decreasing the cysteine:antibody ratio evaluated but increasing the static incubation duration. EDTA addition having no impact on the output parameters, it was decided to replace this additive with Cystine.

### Example 4—Further Optimization

DOE2 was performed using the same conditions as DOE1 but adjusting the factors levels. The other process conditions were based on Example 2 and 3 and Table 2. The wash 3 step was performed by renewing the washing solution 3 times with 1 Column Volume. In between these solution renewals, a static incubation was applied (3 pauses of identical duration).

The evaluated input factor levels are shown in Table 6.

TABLE 6
DOE2 input factors

| | | Lower | Medium | Upper |
|---|---|---|---|---|
| Name | Unit | level | level | level |
| Ratio cysteine:antibody | mol:mol | 10 | 25 | 40 |
| (Cys:mAb) | | | | |
| Duration of the static | h | 4 | 5 | 6 |
| incubation (time) | | | | |
| Ratio Cysteine:Cystine | mM:mM | 0 | 0.05 | 0.1 |

The duration of static incubation was divided equally into three periods of static contact (pauses). For the lower, middle and upper levels, their durations were respectively: 80 minutes; 100 minutes and 120 minutes. The design of experiment was a full factorial design with 3 center points. Two control runs without cysteine washing were performed before and after the set of 11 runs. The experimental design plan and results is given in Table 7.

TABLE 7
Experimental plan of DOE2

| | FACTORS | | | | |
|---|---|---|---|---|---|
| | ratio | | Ratio | Uncystei- | |
| | Cys:mAb | Time | Cysteine:Cystine | nylated | LMW |
| RunOrder | (mol:mol) | (h) | (mol:mol) | species (%) | (%) |
| CTRL | 0 | 0 | 0 | 20.04 | 3.17 |
| initial | | | | | |
| 1 | 40 | 6 | 0.1 | 85.49 | 2.74 |
| 2 | 40 | 4 | 0.1 | 74.71 | 2.93 |
| 3 | 25 | 5 | 0.05 | 76.97 | 2.43 |
| 4 | 40 | 6 | 0 | 80.15 | 2.71 |
| 5 | 25 | 5 | 0.05 | 73.96 | 2.57 |
| 6 | 10 | 6 | 0 | 47.49 | 2.34 |
| 7 | 10 | 6 | 0.1 | 51.51 | 2.43 |
| 8 | 10 | 4 | 0.1 | 44.01 | 2.40 |
| 9 | 40 | 4 | 0 | 73.82 | 3.64 |
| 10 | 25 | 5 | 0.05 | 67.60 | 2.94 |
| 11 | 10 | 4 | 0 | 42.54 | 2.43 |
| CTRL | 0 | 0 | 0 | 21.39 | 3.01 |
| final | | | | | |

In general, the data show that the percentage of un-cysteinylated species was increased for all run conditions, however, some runs had a lower level. The data also show that the LMW level after the step were not closed to control runs (between 2.40 and 3.64%). This treatment resulted in a relative de-cysteinylation of the antibodies (from an average of 20.72% un-cysteinylated to more than 42% and up to 85.5%). Compared to DOE1, these conditions with lower ratio cys:mAb, allowed to limit the increase of the level of low molecular weight species created with the cysteine washing.

FIG. 7 displays the actual by predicted plots for both responses. For the un-cysteinylated species percentage, the R² of the statistical model was 0.95, demonstrating a good model fit. For the LMW % response, the R² was 0.71, meaning that 29% of the variability was not explained by the model.

Table 8 (Effects summary) for both responses confirmed the effects of the most critical factors identified above:ratio cys:mAb and time (h) for the % Uncys. Indeed, the logworth exceeded 2 and p-values were ≤0.01 for these factors. The ratio Cysteine:Cystine was found not significant by the model. The same factors were significant for the other response, LMW %. As the model has a moderate fit, and data suggested that the results might be within the variation of the method compared to the control results. The influence of the factors on LMW % results were not analysed with this DOE2 results. The model did not show an impact of the ratio cysteine:cystine, therefore, it was abandoned. The model did not show interactions between factors. Contrary to DOE1, the most impactful factor on the % Uncysteinylated was the ratio cys:mAb and not the time.

FIG. 8 is a plot of all the runs performed with DOE1 and DOE2. It confirms the effect of the main factors (ratio cysteine:antibody) on the % of uncysteinylated species after the treatment. The data, illustrated in FIG. 8, showed that to reach more than 70% of uncysteinylated species in the eluate, the ratio cys:mAb must be set to at least 25 mol/mol.

### Example 5—Effect of Ratio Cys:mAb and Input Material

Increasing the ratio cysteine:antibody results in an increase of the final percentage of un-cysteinylation species from between 40-50% using a ratio cys:mAb of 10:1 to between 70-90% using a ratio Cys:mAb of 100:1 depending on the static incubation time (FIG. 9). Static incubation duration (time) also increases this percentage.

To conclude on the two DOE studies, the «on-column» Protein A washing with cysteine solution led to de-cysteinylation with potential LMW creation. LMW creation could be limited by using a lower ratio of Cysteine:mAb (less than 40 mol/mol). A ratio Cysteine:antibody of 20 to 40 is preferred (corresponding to 6-13 mM cysteine in wash 3 depending on the ratio and the loading capacity) and a total duration of contact 5 to 6 hours with 3 pauses on column.

Following the same process conditions as described above, four runs were performed using commercially available 5 mL prepacked columns of Praesto® Jetted A50 (Purolite®). Ratio of cysteine:antibody used were 25 and 40, the time of static contact was 6 hours for all four runs with 3 pauses of 120 minutes. Two CHO clarified cell culture fluids (CCCF) were used for this study having different level of uncysteinylated antibodies (CCCF1, around 66.11% uncysteinylated, and CCCF3, 21.30% uncysteinylated).

TABLE 9
Results of Confirmation runs

| | Clarified | Ratio | | |
|---|---|---|---|---|
| Experiment | cell | cysteine:mab | Uncysteinylated | LMW |
| number | culture | (mol:mol) | species (%) | (%) |
| EXP01 | CCCF3 | 40 | 83.28 | 4.11 |
| EXP02 | CCCF1 | 40 | 90.62 | 2.74 |
| EXP03 | CCCF3 | 25 | 80.05 | 2.65 |
| EXP04 | CCCF1 | 25 | 90.51 | 2.24 |

As shown in Table 9, the four experiments led to a de-cysteinylation of the antibody mixture after the on-column treatment. Comparing the data of the two different CCCF showed that with a CCCF having more cysteinylated antibodies (CCCF3), a lower level of uncysteinylated species was observed in the eluates than the experiments run with CCCF1. It is important to take into consideration the initial level of uncysteinylated species in the harvested cell culture to estimate the level of uncysteinylated species reachable with the cysteine treatment. A comparison of EXP01 and EXP03 or EXP02 and EXP04 showed that both ratios 25 or 40 led to similar levels of de-cysteinylation. The percentage of LMW species was higher for EXP01, it was within the method variation for the other experiments. It was not possible to conclude on the impact of the conditions used on LMW creation with this set of data.

### Example 6—Dynamic Incubation with Cysteine Solution During Washing—Study of Number of Pauses and Duration on Fragment Creation and De-Cysteinylation

A study was performed to fine tune the dynamic and static incubations with cysteine during wash 3 of the protein A on-column treatment. Different time of contact were tested: 2 h, 4 h 5 h and 6 h. For each total incubation time, the wash 3 flow was paused to allow static incubation. The number of these pauses was tested.

For this study the ratio cysteine:antibody was set at 40 mol/mol. 8 runs were performed with the parameters shown on Table 10, a duplicate of run 5 h/3 pauses was done.

TABLE 10
Results of the static and dynamic contact duration

| Factors | | | | | |
|---|---|---|---|---|---|
| Static | | Duration | Responses | | |
| incubation | No of | of the | Uncysteinylated | LMW | HMW |
| duration | pauses | pauses | species (%) | (%) | (%) |
| 0 | 0 | 0 | 70.75 | 0.82 | Not |
| | | | | | tested |
| 2 h | 6 | 20 | 85.58 | 56 | Not |
| | pauses | | | | tested |
| 4 h | 6 | 40 | 89.10 | 4.5 | Not |
| | pauses | | | | tested |
| 5 h | 1 | 300 | 86.77 | 3.1 | 4.3 |
| | pause | | | | |
| 5 h | 2 | 150 | 88.00 | 2.4 | 2.3 |
| | pauses | | | | |
| 5 h | 3 | 100 | 81.6 | 2.3 | 1.8 |
| | pauses | | | | |
| 5 h | 3 | 100 | 91.43 | 2.2 | Not |
| | pauses | | | | tested |
| 5 h | 5 | 60 | 90.58 | 32 | Not |
| | pauses | | | | tested |
| 5 h | 15 | 20 | 90.97 | 45 | Not |
| | pauses | | | | tested |
| 6 h | 6 | 60 | 92.38 | 2.4 | Not |
| | pauses | | | | tested |

The results in Table 10 suggest that all the conditions led to a de-cysteinylation of the antibodies (higher uncysteinylated level than the control run without cysteine washing, 70.75%). In general, the data generated by this study confirmed DOE1 and DOE2 observations, meaning that the duration of static contact increased the final level of uncysteinylated species. Looking at data for a same static time (5 h), the final level of uncysteinylated species was not impacted by the number of pauses and their duration. High percentage of LMW were observed for some conditions (>30%), linked to high renewal rate (high number of pauses) and low duration of each pauses. For the runs with 5 h of static incubation, the increase of the pause duration created more aggregates (HMW), 4.3% compared to the run with the standard process of 3 pauses, 1.8%. The results indicated that 3 pauses (3 renewal of washes) for a total static duration of 5 hours was the optimal condition to limit LMW and HMW % creation.

### Example 7—Dynamic Incubation with Cysteine Solution During Washing—Study of Cysteine:Antibody Ratio

The previous studies were done with high level of cysteinylation in the starting material. In order to identify the most appropriate ratio cys:mAb (<40 mol:mol) to de-cysteinylate an IgG mixture with an already high level of uncysteinylated species (around 90%), without impacting the LMW creation, a study was carried out using the same input material and four ratios cys:mAb (10, 20, 30 and 40). This starting material was chosen from clarified cell culture with a high initial level of uncysteinylated species before the on-column treatment (90.5%). An additional study was performed in parallel to compare the pH adjustment after elution to 5.5 or 7.5.

TABLE 11
Results of the ratio Cys:mAb screening or the same initial material

| | | Final | |
|---|---|---|---|
| Ratio | Final pH | Uncysteinylated | Final LMW |
| cys:mAb | adjustment | species (%) | (%) |
| ratio 10 | pH 5.5 | 92.67 | 0.46 |
| mol/mol | pH 7.5 | 92.48 | 0.45 |
| ratio 20 | pH 5.5 | 95.01 | 0.57 |
| mol/mol | pH 7.5 | 94.34 | 0.54 |
| ratio 30 | pH 5.5 | 95.10 | 1.14 |
| mol/mol | pH 7.5 | 94.55 | 0.65 |
| ratio 40 | pH 5.5 | 95.47 | 12.41 |
| mol/mol | pH 7.5 | 95.17 | 8.54 |

The results are displayed in Table 11. The results show that starting from the same material, a ratio cys:mAb below 30 mol/mol resulted in low LMW levels (<1.14%) contrary to the ratio 40, where the LMW % in the eluates were higher than 8.5%. The percentages of uncysteinylated species were close to 95% for all runs above ratio 10. The pH of the eluate did not influence the results and the wash with cysteine solution did not affect the recovery of antibodies during the protein A chromatography step. The samples at pH 7.5 were submitted to deglycosylation and decarboxylation to remove the N-glycans and C-terminal lysines before being analyzed by LC-MS (Table 12).

TABLE 12
Intact Mass (LC-MS) data on four different ratio cys:mAb

| | Peak of mAb containing: | | | | | |
|---|---|---|---|---|---|---|
| | 0 Cysteine 197 | | | Peak of mAb containing: | | |
| | (Uncysteinylated species) | | | 1 Cysteine L97 | | |
| | 147688.68 | | | 147807.82 | | |
| Theoretical mass (Da) | | | Relative | | | Relative |
| Ratios cys:mAb | | Mass error | abundance | | Mass error | abundance |
| (mol/mol) | Mass (Da) | (ppm) | (%) | Mass (Da) | (ppm) | (%) |
| Ratio 10 | 147688.39 | −2.0 | 100 | 147820.38 | 85.0 | 3.6 |
| Ratio 20 | 147688.75 | 0.5 | 100 | 147820.92 | 88.6 | 4.9 |
| Ratio 30 | 147688.63 | −0.3 | 100 | ND⁽¹⁾ | N/A | N/A |
| Ratio 40 | 147689.2 | 3.5 | 100 | ND⁽¹⁾ | N/A | N/A |

The results presented in Table 12 indicate that no cysteinylated forms were detected for ratios cys:mAb of 30 and 40 after the on-column reduction treatment, contrary to ratio 10 and 20. This is illustrated in FIG. 10 where the LC-MS spectra of the experiment at ratio 10 and 30 are represented in mirror.

FIG. 10 is a mirror plot of the product after the cysteine wash treatment using ratio 30 and ratio 10. It shows that contrary to ratio 10, the antibodies are fully decysteinylated when using a ratio cysteine:antibody of 30 mol/mol.

The results of this study suggested that it is preferable to use a ratio 30 to limit LMW generation while maintaining a high de-cysteinylation level.

### CONCLUSION

The optional downstream method of de-cysteinylation developed is based on a standard protein A chromatography step, with an additional wash step containing cysteine at pH 8.0. This process solution leads to de-cysteinylation with potential LMW creation. LMW creation can be limited by using lower ratio of Cysteine:mAb (less than 40 mol:mol) and/or downstream incubation step of the protein A eluate (see Example 7). Any acidic variants and/or glutathionylated species can optionally also be removed via a downstream CEX chromatography step involving an optimized pH wash depending on the loading capacity of antibodies (see Examples 8 to 9).

The preferred process parameters of the on-column de-cysteinylation are summarized in Table 13 below.

TABLE 13
Preferred conditions for on-column de-cysteinylation
using protein A chromatography

| Step | Buffer | CV |
|---|---|---|
| Equilibration | 50 mM Tris-Acetic acid, 150 mM NaCl, pH | 5-10 |
| | 7.4 | |
| Load | Clarified cell culture fluid | N/A |
| | 47 mg/ml (80% DBC) | |
| Wash 1 | 50 mM Tris-Acetic acid, 150 mM NaCl, pH | 5 |
| | 7.4 | |
| Wash 2 | 50 mM Tris-Acetic acid, 1M NaCl, pH 7.4 | 5 |
| Wash 3 | 5-8 mM Tris 6 to 10 mM Cysteine, pH 8.0 | 3 + 1 CV |
| | (ratio cysteine:antibody of 20-30) | between |
| | Total duration of contact: 5 h; 3 pauses | each |
| | of 100 min | pause |
| Wash 4 | 50 mM Sodium Acetate-Acetic acid, pH 5.5 | 5 |
| Elution | 50 mM Sodium acetate-Acetic acid, pH 3.7 | 5 |
| Neutralization | 1M Tris base | N/A |

This method may be adapted depending on initial un-cysteinylated percentage in the protein A load. Based on the data available, the method was efficient on different Protein A resins from different suppliers.

### Example 8—Protein a Eluate Incubation—Study of Holding Time after Protein a Elution and Resultant LMW Level

A run of protein A purification was performed on a 5 mL prepacked column using the procedure displayed in previous sections including the preferred condition with a cysteine solution washing buffer at a ratio 25 mol/mol.

The eluate material after neutralization to pH 7.7 was used as a starting material for a short-term stability study on Low Molecular Weight species evolution during the intermediate product storage. Earlier in the development, it was observed that the LMW % was potentially decreased during the storage. One hypothesis is that Light Chain (LC) and Heavy Light (HHL) fragments spontaneously pair in solution after elution and neutralization of the eluate.

The stability study was conducted at two different temperatures 5° C. (5±3° C.) and 20° C. (20±5° C.). Protein A eluate samples were first stored at the selected temperature and then were frozen at <−70° C. at the time point of interest. In the frame of this stability study, two analytical methods were used: SE-UPLC, for HMW % and Non-Reduced CGE (using a PA800 equipment) for LMW %. Two additional methods, HIC-HPLC and cIEF with CpB treatment were performed on some time points to verify the stability of specific quality attributes (respectively cysteinylation and charge variants).

Results of the stability study are shown in the table below.

TABLE 14
Stability study SEC-UPLC and NR-CGE results of the protein A eluate during a
storage of up to 4 days at −<−70° C., 5 ± 3º C. and 20 ±−5º C.

| | Storage | SE-UPLC | | NR-CGE | | | | |
|---|---|---|---|---|---|---|---|---|
| | before | Total | | | Total | | | Monomer |
| Temperature | freezing | HMW % | Monomer % | Monomer % | LMW % | LC % | HHL % | % |
| <-70° C. | 4 | 0.74 | 99.23 | 98.16 | 1.83 | 0.16 | 0.95 | 98.16 |
| 20 ± 5° C. | 0 | 0.76 | 99.21 | 87.48 | 12.47 | 1.45 | 9.64 | 87.48 |
| | 30 min | 0.77 | 99.2 | 87.36 | 12.6 | 1.48 | 9.73 | 87.36 |
| | 1 h | 0.78 | 99.18 | 90.37 | 9.6 | 1.08 | 7.39 | 90.37 |
| | 2 h | 0.81 | 99.16 | 90.27 | 9.7 | 1.1 | 7.48 | 90.27 |
| | 4 h | 0.86 | 99.11 | 92.59 | 7.41 | 0.82 | 5.66 | 92.59 |
| | 6 h | 0.9 | 99.07 | 94.85 | 5.13 | 0.55 | 3.77 | 94.85 |
| | 24 h | 1.07 | 98.9 | 98.28 | 1.69 | 0.13 | 0.86 | 98.28 |
| | 1, 5 d | 1.11 | 98.85 | 93.61 | 1.62 | 0.12 | 0.8 | 93.61 |
| | 4 d | 1.22 | 98.73 | 98.05 | 1.71 | 0.14 | 0.81 | 98.05 |
| 5 ± 3° C. | 30 min | 0.76 | 99.21 | 81.8 | 12.58 | 1.49 | 9.72 | 81.8 |
| | 1 h | 0.78 | 99.2 | 87.26 | 12.7 | 1.48 | 9.75 | 87.26 |
| | 2 h | 0.78 | 99.19 | 88.61 | 11.36 | 1.3 | 8.8 | 88.61 |
| | 4 h | 0.81 | 99.16 | 89.02 | 10.93 | 1.25 | 8.45 | 89.02 |
| | 6 h | 0.83 | 99.14 | 89.54 | 10.43 | 1.2 | 8.05 | 89.54 |
| | 24 h | 0.91 | 99.06 | 97.1 | 2.88 | 0.29 | 1.82 | 97.1 |
| | 1, 5 d | 0.92 | 99.05 | 97.96 | 2.01 | 0.16 | 1.13 | 97.96 |
| | 4 d | 1.16 | 98.81 | 98.19 | 1.63 | 0.12 | 0.82 | 98.19 |

The results presented in Table 14 suggest a slight increase of HMW over time from 0.8% to 1.2% in 4 days at both evaluated temperatures, and no increase when stored at <−70° C. A decrease of the total LMW % by NR-CGE was observed from 12.5% to 1.6% at 20±5° C. in 1.5 days and to 1.63% at 5±3° C. in 4 days. The cIEF and HIC-HPLC results remained stable during the holding time.

FIG. 11 displays the results of Table 14 and shows the decrease of total percentage of LMW in the protein A eluate over a period of 4 days of holding time. HHL and LC fragments are the most affected by the decrease suggesting a reversibility of the fragments caused by the cysteine wash during protein A chromatography.

These observations support the hypothesis of a re-oxidation of the HHL and LC species bonds to monomer during storage in neutral conditions (here pH 7.7). These data demonstrate that if the protein A eluate is stored at room temperature for 24 h after de-cysteinylation, the level of induced low molecular weight species decreases significantly.

### Example 9—Study of a CEX or MMC Process to Reduce Acidic Variants Level and to Remove Glutathionylated Species

Secukinumab were purified using a first capture step of protein A chromatography with cysteine washing as described in previous sections, followed by an Anion exchange chromatography in Flow-through mode. The quality profile of this product was analysed by several methods and showed important level of acidic variants (30 to 40% depending on the experiment) and some glutathionylation. Glutathionylation consists in the reversible addition of a proximal donor of glutathione to thiolate anions of cysteine residues in secukinumab. Glutathionylated species are linked to the non-paired cysteine in secukinumab's light chain CDR3.

Secukinumab was further purified using a CEX or MMC chromatography using the process described in Table 15 below, the resins used were either Capto S impact or Capto MMC impact (Cytiva).

The loaded product was adjusted to the same pH and ionic strength as the column equilibration buffer (Table 15) and the column was loaded at 10 g_{IgG}/mLᵣₑₛᵢₙ, therefore loading pH target was 5.5±0.1 and loading conductivity target was ≤5.0 mS/cm. The column was then washed with Sodium Phosphate buffer at a pH above the pi of each secukinumab variants (pH 8.9±0.1) preceded and followed by a step at pH 7.4±0.1. Following analytical methods were used: Non-Reduced CGE (using a PA800 equipment) for LMW, HIC-HPLC for cysteinylation level, cIEF with CpB for acidic variants evaluation and intact mass spectrometry for glutathionylation level.

Data presented in Table 15 are an average of the results obtained on 6 runs following these conditions.

TABLE 15
Data showing Secukinumab purification using the described CEX process.

| | | | | | Acidic | Glutathionylation |
|---|---|---|---|---|---|---|
| | | Recovery | Uncysteinylation | Total | variants | relative |
| Chromatographic | | (%) | (%) | LMW (%) | (%) | area (%) |
| step | Solution | n = 6 | n = 6 | n = 6 | n = 6 | n = 5 |
| Equilibration | 50 mM Na- | | | | | |
| | Acetate-HAc, | | | | | |
| | pH5.5 | | | | | |
| Load | PD-AEX-06 | N/A | 93.2 | 2.12 | 36.2 | 4.5 |
| | adjusted pH 5.5 | | | | | |
| Wash pH 7.4 | 50 mM Tris pH | | | | | |
| | 7.4 | | | | | |
| Wash basic | 50 mM Tris pH | 21.3 | 75.9 | 4.4 | 94.8 | 60.8 |
| | 8.9 | | | | | |
| Wash pH 7.4 | 50 mM Tris pH | | | | | |
| | 7.4 | | | | | |
| Re Equilibration | 50 mM Na- | | | | | |
| | Acetate-HAc, | | | | | |
| | pH5.5 | | | | | |
| Elution | 50 mM Na- | 67.8 | 95.3 | 0.84 | 19.7 | 0.5 |
| | Acetate-HAc, | | | | | |
| | 500 mM NaCl | | | | | |
| | pH5.5 | | | | | |

These data demonstrate that a washing at high pH (pH=8.9±0.1) during the CEX (Cation Exchange Chromatography) enables to reduce efficiently and significantly the acidic variants of secukinumab (from 36.2% to 19.7%) and the glutathionylated species (from 4.5% to 0.5%), as well as reducing the level of cysteinylated species and low molecular weight species.

It illustrates that the best purity of secukinumab can be achieved when the washing buffer pH is at a high pH (here pH=8.9 for a 10 mg/mL loading capacity), but as high as possible without washing secukinumab off the column.

### Example 10—Increasing Loading Density of the CEX while Keeping the Acidic and Glutathionylated Species Clearance

Loading density and pH of the wash were screened on a CEX Capto S Impact 1 mL prepacked column. CEX were performed with different conditions: four different loading densities: 20, 30, 40 and 50 g/L. For each of these loading densities, different pH were applied for the post loading wash buffer among: 7.4, 7.6, 7.8 and 8.0.

The chromatography buffers were the same as the previous example without the pH 7.4 washes. The basic washing was 3 CV and the loading material were antibodies purified using a first capture step of protein A chromatography with cysteine washing as described in previous sections, followed by an Anion exchange chromatography in Flow-through mode, adjusted at pH 5.5.

The factors watched for this screening study were the yield (%) and the acidic variant removal, measured using cIEF with CpB treatment. Briefly, the sample is diluted at 1 mg/mL and digested with Carboxypeptidase B. After a buffer exchange, sample is diluted at 1 mg/mL and mix with an ampholytes MasterMix before analysis on a Maurice equipment (Proteinsimple). Charged variants species are separated based on their pI, during 9 minutes focusing time.

The Table below summarizes the runs performed and results obtained for the yield and the acidic variant removal.

TABLE 16
Data of CEX runs performed on a 1 mL columns at different loading
densities and pH of post-loading wash

| | | | | | | Acidic | |
|---|---|---|---|---|---|---|---|
| | Acidic | | | | Acidic | variant | Removal |
| | variant in | Loading | | | variant in | in the | of acidic |
| | the load | density | Washing | | the wash | eluate | variant |
| Run | (%) | (g/L) | buffer pH | Yield (%) | (%) | (%) | (%) |
| PD-CEX-28 | 48.8 | 20 | 7.6 | 73.7 | 94.5 | 46.2 | 5 |
| PD-CEX-29 | 48.8 | 20 | 7.8 | 74.2 | 86.0 | 43.1 | 12 |
| PD-CEX-30 | 48.8 | 20 | 8.0 | 34.0 | 60.8 | 34.9 | 29 |
| PD-CEX-32 | 51.1 | 30 | 7.8 | 73.9 | 90.6 | 47.7 | 7 |
| PD-CEX-33 | 51.1 | 30 | 8.0 | 59.3 | 72.2 | 40.2 | 21 |
| PD-CEX-35 | 51.7 | 40 | 7.6 | 63.2 | 77.2 | 43.3 | 16 |
| PD-CEX-36 | 51.7 | 40 | 7.8 | 50.5 | 68.7 | 40.7 | 21 |
| PD-CEX-38 | 50.5 | 50 | 7.6 | 46.3 | 62.9 | 42.9 | 15 |
| PD-CEX-39 | 50.5 | 50 | 7.8 | 42.0 | 60.7 | 39.4 | 22 |

The results show that, for a same loading density (ex. 20 g/L), the removal of acidic variants increases with the pH of the post loading washing applied, while the yield decreases.

Based on the results of these runs, runs at 20 and 30 g/L loading densities were performed on a 5 mL prepacked column and on Capto MMC Impres resin. For these assays, the buffers used were the same as for the previous example with a pre- and post-washes at pH 7.4. The table below summarizes the runs performed on the 5 mL column.

TABLE 17
Data obtained for the runs on a 5 mL prepacked column at 20 and
30 g/L loading density

| | Acidic | | | | Acidic | Acidic | |
|---|---|---|---|---|---|---|---|
| | variant | | Washing | | variant | variant | Removal |
| | in the | Loading | buffer | | in the | in the | of acidic |
| | load | density | pH | | wash | eluate | variant |
| Resin | (%) | (g/L) | (3 CV) | Yield (%) | (%) | (%) | (%) |
| Capto S Impact | 46.6 | 20 | 7.4-7.8-7.4 | 71.8 | 98.2 | 38.3 | 18 |
| Capto S Impact | 46.6 | 20 | 7.4-8.0-7.4 | 65.8 | 97.5 | 33.5 | 28 |
| Capto S Impact | 45.1 | 20 | 7.4-8.2-7.4 | 58.4 | 95.8 | 28.6 | 37 |
| Capto S Impact | 45.1 | 30 | 7.4-8.0-7.4 | 51.6 | 72.2 | 26.0 | 42 |
| Capto S Impact | 45.1 | 30 | 7.4-7.8-7.4 | 66.0 | 91.4 | 31.0 | 31 |
| Capto MMC | 40.6 | 30 | 7.5 | 66.3 | 69.7 | 32.0 | 21 |
| Impres | | | | | | | |

According to the results, at 20 g/L and 30 g/L the best conditions to increase the removal of acidic variants with an acceptable yield were found to be at pH 8.0 and 8.2 for the CEX and 7.5 with the MMC. The condition at a loading capacity of 30 g/L with a pH wash sequence of 7.4-8.0-7.4 was chosen for a scale up on a 20 cm bed height manually packed column at 5 minutes residence time. Table below summarized the result obtained for one of the runs with the selected conditions.

TABLE 18
Data showing Secukinumab purification using the described CEX process

| | | | | | | Gluta- |
|---|---|---|---|---|---|---|
| | | | Uncysteinylation | | Acidic | thionylation |
| Chromatographic | | Recovery | relative | Total | variants | relative |
| step | Solution | (%) | area (%) | LMW (%) | (%) | area (%) |
| Equilibration | 50 mM Na- | | | | | |
| | Acetate-HAc, | | | | | |
| | pH5.5 | | | | | |
| Load (30 g/L) | | N/A | 92.31 | 2.7 | 42.7 | 5.4 |
| Wash pH 7.4 | 50 mM Tris pH 7.4 | | | | | |
| Wash basic | 50 mM Tris pH 8.0 | 21.2 | 86.83 | 8.3 | 91.01 | 63.5 |
| Wash pH 7.4 | 50 mM Tris pH 7.4 | | | | | |
| Re Equilibration | 50 mM Na- | | | | | |
| | Acetate-HAc, | | | | | |
| | pH5.5 | | | | | |
| Elution | 50 mM Na- | 75.6 | 94.13 | 1.2 | 29.0 | 1.8 |
| | Acetate-HAc, 500 | | | | | |
| | mM NaCl pH5.5 | | | | | |

In this example, the acidic variants were reduced by 32%.

These data demonstrate that a washing at high pH during the CEX (Cation Exchange Chromatography) enables to reduce efficiently and significantly the acidic variants of secukinumab as well as the cysteinylated and glutathionylated species, and low molecular weight species. It also demonstrates the interaction between two critical process parameters: the loading density and the pH of this washing step on the process performance (impurities clearance and yield).

It illustrates that the best purity of secukinumab can be achieved when the washing buffer pH is as high as possible without washing secukinumab off the column. For example, pH=8.0±0.05 for a loading density of 30 g/L.

1.-50. (canceled)

51. A method of selectively reducing one or more unpaired cysteines of a recombinant monoclonal antibody during production of the antibody, wherein the method comprises:
(a) providing a cell capable of recombinant expression of the antibody;
(b) culturing the cell in a cell culture medium, wherein the cells are cultured at a first temperature and then shifted to a second temperature, wherein the second temperature is lower than the first temperature,
wherein the cells are maintained in culture until at least 90% or more of the unpaired cysteines are de-cysteinylated; and
(c) harvesting the antibodies from the cell culture to obtain a preparation of the antibody.

52. The method of claim 51, wherein the cysteine is in the complementary determining region (CDR) of the antibody and/or wherein the antibody is an anti-IL-17 antibody.

53. The method of claim 51, wherein the antibody comprises:
(a) a VH sequence having at least about 90% or more sequence identity to the amino acid sequence of SEQ ID NO: 3; and
(b) a VL sequence having at least about 90% or more sequence identity to the amino acid sequence of SEQ ID NO: 4.

54. The method of claim 51, wherein the antibody is secukinumab, biosimilar or variant thereof, and/or the cell is a eukaryotic cell, optionally a CHO cell.

55. The method of claim 51, wherein the method comprises selectively reducing light chain (LC) Cys97.

56. The method of claim 51, wherein:
(a) the pH of the cell culture is maintained at a constant level, optionally wherein the cell culture is maintained at a pH of between about 6.7 to about 7.1 and/or the pH of the cell culture is maintained until at least 90% or more of the unpaired cysteines are de-cysteinylated;
(b) the second temperature is between about 3° C. to about 5° C. lower than the first temperature, optionally wherein the second temperature is about 4° C. lower than the first temperature, optionally wherein the first temperature is about 37° C., further optionally wherein the second temperature is about 33° C.; and/or
(c) wherein the cells are:
(i) cultured at the first temperature for between about 8 days to about 13 days before culturing the cells at the second temperature, optionally wherein the cells are cultured for about 10 days before culturing the cells at the second temperature; and/or
(ii) maintained in culture for between about 14 days to about 17 days.

57. The method of claim 51, wherein step (b) further comprises:
(i) stirring or agitating the cell culture at a rate of about 160 to about 180 rpm (e.g., 170 rpm);
(ii) maintaining a dissolved oxygen (DO) concentration of between about 20% to about 50%, optionally wherein the DO concentration is between about 30% to about 40%;
(iii) inoculating the cell culture medium with the cells at a seeding cell density of between about 0.2×10⁶ cells/ml to about 0.6×10⁶ cells/ml, optionally wherein the seeding cell density is about 0.4×10⁶ cells/ml;
(iv) supplementing the cell culture medium with cell feed, optionally wherein the cell culture medium is supplemented with cell feed each day from about day 2, 3 or 4 of the culture to the penultimate day of the culture;
(v) addition of supplemental glucose to the cell culture medium to a concentration between about 2 g/L to about 7 g/L; and/or
(vi) addition of an antifoam emulsion, optionally wherein the antifoam emulsion is a silicone antifoam emulsion.

58. The method of claim 51, wherein:
(i) the cell culture medium is a chemically defined medium and/or animal-component free, optionally wherein the medium is supplemented with a mannosidase I inhibitor, optionally wherein the mannosidase I inhibitor is Kifunensine, further optionally wherein the Kifunensine is present in the cell culture medium at a concentration of less than about 5 μg/kg; and/or
(ii) the antibodies are harvested from the cell culture by centrifugation, flocculation, depth filtration and/or tangential flow filtration;

59. The method of claim 51, wherein the method further comprises:
(d) passing the harvested antibodies through one or more chromatography material(s), optionally wherein the chromatography material is cation-exchange (CEX) material, thereby obtaining a purified preparation of antibodies.

60. The method of claim 51, wherein the method further comprises:
(d) passing a sample of the harvested antibodies through one or more chromatography material(s), thereby binding the antibodies to the chromatography material(s), optionally wherein the chromatography material is cation-exchange (CEX) material;
(e) contacting the bound antibodies with a wash buffer, wherein the wash buffer is at a pH above about 7.0, optionally wherein the wash buffer is a Tris buffer; and
(f) eluting the antibodies from the chromatography material(s), optionally wherein the eluting comprises passing an elution buffer through the chromatography material, wherein the elution buffer is at a pH of about 5.5.

61. The method of claim 59, wherein:
(i) about 10 g/L of antibodies or less are loaded onto the chromatography material(s), and the wash buffer is at a pH of about 8.8 to about 9.0; or
(ii) more than about 10 g/L of antibodies are loaded onto the chromatography material(s), and the wash buffer is at a pH of about 7.0 to about 8.4, optionally about 8.0.

62. The method of claim 59, wherein:
(i) the chromatography material(s) are washed with one or more equilibration buffer(s) and/or loading buffer(s) prior to step (e);
(ii) the bound antibodies are contacted with one or more pre-wash buffers prior to step (e);
(iii) the bound antibodies are contacted with one or more post-wash buffers after step (e); and/or
(iv) the bound antibodies are contacted with one or more re-equilibration buffers after step (e);
optionally wherein:
(a) the equilibrium buffer(s), loading buffer(s) and/or re-equilibrium buffer(s) are at a pH of about 5.5; and/or (b) the pre- and/or post-wash buffers are at pH of about 7.4.

63. A method of removing acidic variants and/or glutathionylation from a sample of antibodies, wherein the method comprises:
(a) passing the antibodies through one or more chromatography material(s), thereby binding the antibodies to the chromatography material(s), optionally wherein the chromatography material is CEX material;
(b) contacting the bound antibodies with a wash buffer, wherein the wash buffer is at a pH of above about 7.0, optionally wherein the buffer is a Tris buffer; and
(c) eluting the antibodies from the chromatography material, optionally wherein the eluting comprises passing an elution buffer through the chromatography material,
wherein the elution buffer is at a pH of about 5.5.

64. The method of claim 63, wherein:
(i) the antibodies are anti-IL-17 antibodies;
(ii) the antibodies comprise:
(a) a VH sequence having at least about 90% or more sequence identity to the amino acid sequence of SEQ ID NO: 3; and
(b) a VL sequence having at least about 90% or more sequence identity to the amino acid sequence of SEQ ID NO: 4; and/or
(iii) the antibodies are secukinumab or a biosimilar or variant thereof.

65. The method of claim 63, wherein the sample of antibodies is obtained by a method according to claim 1

66. The method of claim 63, wherein:
(i) about 10 g/L of antibodies or less are loaded onto the chromatography materials(s), and the wash buffer is at a pH of about 8.8 to 9.0; or
(ii) more than about 10 g/L of antibodies are loaded onto the chromatography material(s), and the wash buffer is at a pH of about 7.0 to 8.4, optionally about 8.0.

67. The method of claim 63, wherein:
(i) the chromatography material is washed with one or more equilibration buffers and/or loading buffers prior to step (b);
(ii) the bound antibodies are contacted with one or more pre-wash buffers prior to step (b);
(iii) the bound antibodies are contacted with one or more post-wash buffers after step (b); and/or
(iv) the bound antibodies are contacted with one or more re-equilibration buffers after step (b);
optionally wherein:
(a) the equilibrium buffers, loading buffers and/or re-equilibration buffers are at a pH of about 5.5; and/or
(b) the pre- and/or post-wash buffers are at pH of about 7.4.

68. The method of claim 51, wherein:
(a) at least about 90%, 95% or more of the unpaired cysteines of the harvested, eluted and/or further purified antibodies are un-cysteinylated; and/or
(b) about 98.5% or more of the harvested, eluted and/or further purified antibodies are intact; and/or
(c) the eluted and/or further purified antibodies retain biological activity and/or structure as compared to a reference approved antibody, optionally wherein the harvested, eluted and/or purified antibodies have:
(i) a total % LMW of less than about 1.5%;
(ii) a % amount of acidic variants of less than about 25%; and/or
(iii) a glutathionylation relative area (%) of less than about 1.5%.

69. A purified preparation of antibodies obtainable by the method of claim 51.

70. A purified preparation of secukinumab, biosimilar or variant thereof, wherein:
(i) LC Cys97 is un-cysteinylated in at least 90%, 95%, 96%, 97%, 98%, 99% or more of the antibodies;
(ii) the % level of intact antibodies is at least about 98.5%, 99.0%, 99.9% or more; and/or
(iii) the antibodies retain biological activity and/or structure as compared to a reference approved antibody.


SEQUENCE LISTING (10 sequences)

SEQ ID NO: 1 - protein, 457 aa, Artificial Sequence
  Secukinumab Heavy Chain (full length)
EVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 215 aa, Artificial Sequence
  Secukinumab Light Chain (full length)
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 120 aa, Artificial Sequence
  Secukinumab Heavy Chain (variable)
EVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRG

SEQ ID NO: 4 - protein, 113 aa, Artificial Sequence
  Secukinumab Light Chain (variable)
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRTVAA

SEQ ID NO: 5 - protein, 5 aa, Artificial Sequence
  Secukinumab Heavy Chain CDR1
NYWMN

SEQ ID NO: 6 - protein, 19 aa, Artificial Sequence
  Secukinumab Heavy Chain CDR2
AINQDGSEKYYVGSVKGRF

SEQ ID NO: 7 - protein, 17 aa, Artificial Sequence
  Secukinumab Heavy Chain CDR3
DYYDILTDYYIHYWYFD

SEQ ID NO: 8 - protein, 12 aa, Artificial Sequence
  Secukinumab Light Chain CDR1
RASQSVSSSYLA

SEQ ID NO: 9 - protein, 7 aa, Artificial Sequence
  Secukinumab Light Chain CDR2
GASSRAT

SEQ ID NO: 10 - protein, 10 aa, Artificial Sequence
  Secukinumab Light Chain CDR3
QQYGSSPCTF